SEQUENCE LISTING (44 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT525
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 12 aa, Artificial Sequence
  HisTag
MHHHHHHSSGSS

SEQ ID NO: 12 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 13 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 14 - protein, 576 aa, Artificial Sequence
  PRT525
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 623 aa, Artificial Sequence
  PRT720
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 23 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 24 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 25 - protein, 593 aa, Artificial Sequence
  Met-PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 26 - protein, 590 aa, Artificial Sequence
  Met-PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 27 - protein, 593 aa, Artificial Sequence
  Met-PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 28 - protein, 590 aa, Artificial Sequence
  Met-PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 29 - protein, 590 aa, Artificial Sequence
  Met-PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 30 - protein, 565 aa, Artificial Sequence
  Met-PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 31 - protein, 565 aa, Artificial Sequence
  Met-PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 32 - protein, 1179 aa, Artificial Sequence
  Met-PRT966
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 33 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 34 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 35 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 36 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 37 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 38 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 39 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 40 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 41 - protein, 590 aa, Artificial Sequence
  Met-PRT917
MGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 42 - protein, 587 aa, Artificial Sequence
  Met-PRT1028
MGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

SEQ ID NO: 43 - protein, 601 aa, Artificial Sequence
  PRT917
MHHHHHHSSGSSGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 44 - protein, 598 aa, Artificial Sequence
  PRT1028
MHHHHHHSSGSSGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

(19) United States
(12) Patent Application Publication
(10) Pub. No.: US 2022/0235099 A1
(43) Pub. Date: Jul. 28, 2022

(54) Structural Protein Microbody and Method for Producing Same, Method for Producing Nanofiber, and Method for Producing Protein Structure

(71) Applicant: Spiber Inc., Tsuruoka-shi, Yamagata (JP)

(72) Inventors: Hironari Kamikubo, Ikoma-shi, Nara (JP); Yugo Hayashi, Ikoma-shi, Nara (JP); Takehiro Sato, Tsuruoka-shi, Yamagata (JP)

(73) Assignee: Spiber Inc., Tsuruoka-shi, Yamagata (JP)

(21) Appl. No.: 17/614,058

(22) PCT Filed: May 28, 2020
(86) PCT No.: PCT/JP2020/021171

§ 371 Date: Nov. 24, 2021

(30) Foreign Application Priority Data
May 29, 2019 (JP) 2019-100615
May 29, 2019 (JP) 2019-100616
May 29, 2019 (JP) 2019-100617

(51) Int. Cl. C07K 14/435; D01F 4/02

(52) U.S. Cl. CPC C07K 14/43518; C07K 2299/00; D01F 4/02

(57) ABSTRACT

Provided is a structural protein microbody that functions as a core for forming a protein nanofiber. There is provided a structural protein microbody including a structural protein, in which the structural protein microbody satisfies at least two of the following (i) to (iii): (i) a peak is present within a range of 480 to 500 nm in a fluorescence intensity measurement by thioflavin T staining; (ii) a peak is present in a region where Q is 0.15 or less in a modified Kratky plot of small angle X-ray scattering (SAXS); and (iii) the structural protein microbody is an aggregate of two or more structural protein molecules.

### TECHNICAL FIELD

The present invention relates to a structural protein microbody and a method for producing the same, a method for producing a nanofiber, and a method for producing a protein structure.

### BACKGROUND ART

A nanostructure obtained by using metal molecules has been put into practical use or has been almost put into practical use in a dye sensitized solar cell (titanium oxide), a conductive ink (silver nanowire), or the like.

The nanostructure has been spotlighted also in the field of biotechnology, and a protein nanofiber has been expected to be used for a cell scaffolding sheet whose mechanical properties are designed as desired, a biomolecular device, a cell engineering device, a regenerative medicine and tissue engineering, or a biosensor and actuator, and as a lightweight and high-strength material, a green nanohybrid, an environmental purification material, a self-healing material, a filter, or a material for high-precision equipment related to spinning, coating, or structural and physical property analysis.

However, a protein nanofiber has not been put into practical use (Non Patent Literature 1).

In addition, some highly oriented polymer materials formed of nanofibers exhibit excellent physical properties such as conductivity, thermal conductivity, and wear resistance, and such materials are extremely useful.

For example, a high degree of orientation of a natural yarn produced by a spider is confirmed by measurement with an atomic force microscope (AFM). However, it is difficult to artificially impart such a high degree of orientation to a protein nanofiber.

### CITATION LIST

### Non Patent Literature

Non Patent Literature 1: L. Wang, Y. Sun, Z. Li, A. Wu, and G. Wei, Materials (Basel), vol. 9, no. 1, 2016 “Bottom-up synthesis and sensor applications of biomimetic nanostructures”

### SUMMARY OF INVENTION

### Technical Problem

The present invention has been made in view of the above circumstances, and a first object of the present invention is to provide a structural protein microbody that functions as a core for forming a protein nanofiber.

A second object of the present invention is to provide a method capable of advantageously producing the structural protein microbody having the characteristics described above.

A third object of the present invention is to provide a method for producing a nanofiber capable of easily producing a nanofiber composed of a protein.

A fourth object of the present invention is to provide a method for producing a protein structure capable of producing a structure in which a plurality of protein nanofibers are highly oriented.

### Solution to Problem

The present invention for implementing the first object (first invention) relates to, for example, the following inventions.

[1-1] A structural protein microbody including a structural protein, in which the structural protein microbody satisfies at least two of the following (i) to (iii): (i) a peak is present within a range of 480 to 500 nm in a fluorescence intensity measurement by thioflavin T staining; (ii) a peak is present in a region where Q is 0.15 or less in a modified Kratky plot of small angle X-ray scattering (SAXS); and (iii) the structural protein microbody is an aggregate of two or more structural protein molecules.

Such a structural protein microbody functions as a core for forming a protein nanofiber. Therefore, a protein nanofiber can be easily formed, for example, by adding the structural protein microbody to a protein solution.

[1-2] The structural protein microbody according to [1-1], in which the structural protein microbody satisfies all of (i) to (iii).

[1-3] The structural protein microbody according to [1-1] or [1-2], in which an average particle size measured by a dynamic light scattering method is 1 to 50 nm.

[1-4] The structural protein microbody according to any one of [1-1] to [1-3], in which the structural protein microbody satisfies at least (ii), and a magnitude of the peak is 1.1 times or more greater than an average value in a region where Q is 0.15 or more and 0.3 or less in the modified Kratky plot of small angle X-ray scattering (SAXS).

[1-5] The structural protein microbody according to any one of [1-1] to [1-4], in which the structural protein microbody satisfies at least (iii), and an origin scattering intensity normalized by a weight concentration obtained by Guinier analysis is 1.5 times or more greater than an origin scattering intensity of non-aggregated structural protein molecules.

[1-6] The structural protein microbody according to any one of [1-1] to [1-5], in which the structural protein contains modified fibroin.

[1-7] The structural protein microbody according to [1-6], in which the structural protein contains modified spider silk fibroin.

The present invention for implementing the second object (second invention) relates to, for example, the following inventions.

[2-1] A method for producing a structural protein microbody, the method including: a first step of obtaining a structural protein solution containing a structural protein and a solubilizing agent; and a second step of reducing solubility of the structural protein in the structural protein solution to form the structural protein microbody according to any one of [1-1] to [1-7].

According to such a production method, a structural protein microbody that functions as a core for forming a protein nanofiber can be easily produced.

[2-2] The method for producing a structural protein microbody according to [2-1], in which the second step is a step of reducing the solubility by at least one method selected from the group consisting of temperature adjustment, addition of water, addition of a surfactant, addition of an organic solvent, and addition of an inorganic salt.

[2-3] The method for producing a structural protein microbody according to [2-2], in which the second step is a step of reducing the solubility by two or more methods selected from the group consisting of temperature adjustment, addition of water, addition of a surfactant, addition of an organic solvent, and addition of an inorganic salt.

[2-4] The method for producing a structural protein microbody according to [2-1], in which the second step is a step of reducing the solubility by applying a shear stress to the structural protein solution.

[2-5] The method for producing a structural protein microbody according to any one of [2-1] to [2-4], in which the solubilizing agent contains at least one selected from the group consisting of dimethyl sulfoxide, 1,1,1,3,3,3-hexafluoro-2-propanol, guanidine hydrochloride (GuHCl), guanidine thiocyanate, sodium iodide, and perchlorate.

[2-6] The method for producing a structural protein microbody according to any one of [2-1] to [2-5], further including a third step of collecting the structural protein microbody by centrifugation.

[2-7] The method for producing a structural protein microbody according to any one of [2-1] to [2-6], in which the structural protein contains modified fibroin.

[2-8] The method for producing a structural protein microbody according to [2-7], in which the structural protein contains modified spider silk fibroin.

The present invention for implementing the third object (third invention) relates to, for example, the following inventions.

[3-1] A method for producing a nanofiber, the method including: step A of preparing a protein solution in which a protein is dissolved; and step B of mixing the protein solution with the structural protein microbody according to any one of [1-1] to [1-7] to obtain a protein nanofiber.

In such a production method, the protein solution is mixed with the structural protein microbody, such that the protein can be self-organized using the structural protein microbody as a core and a nanofiber composed of a protein can be easily formed.

[3-2] The method for producing a nanofiber according to [3-1], in which the protein solution contains a first solvent, and the first solvent is one selected from the group consisting of an organic solvent, a salt solution, an acidic solution, a basic solution, and a chaotropic solution.

[3-3] The method for producing a nanofiber according to [3-2], in which the first solvent is one selected from the group consisting of an organic solvent, a salt solution, an acidic solution, and a basic solution.

[3-4] The method for producing a nanofiber according to [3-3], in which the first solvent is one selected from the group consisting of 1,1,1,3,3,3-hexafluoro-2-propanol and dimethyl sulfoxide.

[3-5] The method for producing a nanofiber according to any one of [3-1] to [3-4], in which the protein includes a structural protein.

[3-6] The method for producing a nanofiber according to [3-5], in which the structural protein contains modified fibroin.

[3-7] The method for producing a nanofiber according to [3-6], in which the structural protein contains modified spider silk fibroin.

The present invention for implementing the fourth object (fourth invention) relates to, for example, the following inventions.

[4-1] A method for producing a protein structure, the method including: step (a) of preparing a structural precursor containing a fibrous substance containing a protein; and step (b) of orienting the fibrous substance in one direction by applying an anisotropic stress to the structural precursor to obtain the protein structure, in which the fibrous substance contains at least one of the structural protein microbody according to any one of [1-1] to [1-7] and a protein nanofiber.

According to such a production method, a protein structure in which a protein nanofiber is highly oriented can be easily produced.

[4-2] The method for producing a protein structure according to [4-1], in which the protein nanofiber is formed by self-organizing the protein using the structural protein microbody as a core.

[4-3] The method for producing a protein structure according to [4-1] or [4-2], in which the protein nanofiber has an amyloid-like crystal.

[4-4] The method for producing a protein structure according to [4-3], in which the amyloid-like crystal is oriented perpendicular to an orientation direction of the fibrous substance.

[4-5] The method for producing a protein structure according to any one of [4-1] to [4-4], in which a thickness of the fibrous substance is 3 nm or more.

[4-6] The method for producing a protein structure according to any one of [4-1] to [4-5], in which in step (b), the anisotropic stress is applied by fixing both ends of the structural precursor in one direction and drying and shrinking the structural precursor.

[4-7] The method for producing a protein structure according to any one of [4-1] to [4-6], in which the structural precursor is at least one selected from the group consisting of a hydrogel, a fiber, and a film.

[4-8] The method for producing a protein structure according to any one of [4-1] to [4-7], in which the protein contains modified fibroin.

[4-9] The method for producing a protein structure according to [4-8], in which the protein contains modified spider silk fibroin.

### Advantageous Effects of Invention

According to the first invention, a structural protein microbody that functions as a core for forming a protein nanofiber can be provided.

According to the second invention, a method for advantageously producing a structural protein microbody that functions as a core for forming a protein nanofiber can be provided.

According to the third invention, a method for producing a nanofiber capable of easily producing a nanofiber composed of a protein can be provided.

According to the fourth invention, a method for producing a protein structure capable of producing a structure in which a plurality of protein nanofibers are highly oriented can be provided.

### BRIEF DESCRIPTION OF DRAWINGS

FIG. 1 is a schematic view illustrating a change of a structure of a protein, in which (a) illustrates a dissolved protein, and (b) illustrates a protein in which columnar nanofibers are formed.

FIG. 2 is a view for describing a measurement principle of a SAXS measurement.

FIG. 3 is a diagram illustrating an example of a result of a fluorescence intensity measurement of a structural protein microbody by ThT staining.

FIG. 4 is a diagram illustrating an example of a modified Kratky plot of a structural protein microbody.

FIG. 5 is a schematic view illustrating an example of a domain sequence of modified fibroin.

FIG. 6 is a diagram illustrating a distribution of values of z/w (%) in naturally derived fibroin.

FIG. 7 is a diagram illustrating a distribution of values of x/y (%) in naturally derived fibroin.

FIG. 8 is a schematic view illustrating an example of a domain sequence of modified fibroin.

FIG. 9 is a schematic view illustrating an example of a domain sequence of modified fibroin.

FIG. 10 is a diagram illustrating an example of a result of a fluorescence intensity measurement for confirming formation of a nanofiber.

FIG. 11 is a diagram illustrating another example of a result of a fluorescence intensity measurement for confirming formation of a nanofiber.

FIG. 12(a) is a view for describing a step of producing a protein structure of Example 4, and FIG. 12(b) is a view for describing a step of producing a protein structure of Comparative Example 2.

FIG. 13 is a view illustrating a two-dimensional X-ray diffraction profile of the protein structure of Example 4.

FIG. 14 is a view illustrating a two-dimensional X-ray diffraction profile of the protein structure of Comparative Example 2.

FIG. 15(a) is a view illustrating an AFM image of the protein structure of Example 4, and FIG. 15(b) is a view illustrating an AFM image of the protein structure of Comparative Example 2.

### DESCRIPTION OF EMBODIMENTS

Hereinafter, embodiments of the present invention will be described in detail. However, the present invention is not limited to the following embodiments.

(Structural Protein Microbody)

A structural protein microbody according to the present embodiment includes a protein and satisfies at least two (preferably all three) of the following (i) to (iii), (i) a peak is present within a range of 480 to 500 nm in a fluorescence intensity measurement by thioflavin T staining; (ii) a peak is present in a region where Q is 0.15 or less in a modified Kratky plot of small angle X-ray scattering (SAXS); and (iii) the structural protein microbody is an aggregate of two or more structural protein molecules.

The structural protein microbody according to the present embodiment functions as a core for forming a protein nanofiber. Therefore, a protein nanofiber can be easily formed, for example, by mixing the structural protein microbody according to the present embodiment with a protein solution. Hereinafter, (i) to (iii) will be described in detail.

<(i) Fluorescence Intensity Measurement by Thioflavin T Staining (ThT Staining)>

Thioflavin T (ThT) is a fluorescent dye that strongly reacts with a β-sheet structure. The presence or absence of the β-sheet structure in the structural protein microbody can be confirmed by staining the structural protein microbody with ThT and measuring a fluorescence intensity.

The fluorescence intensity can be measured by a fluorometer. An example of the fluorometer can include JASCO FP-8200 (manufactured by JASCO Corporation). The measurement may be performed according to the manual attached to the device.

Specifically, the fluorescence intensity can be measured under the following conditions. A measurement sample obtained by dispersing the structural protein microbody in a dispersion (aqueous solution of 6 M urea, 10 mM trishydroxymethylaminomethane hydrochloride (TrisHCl), and 5 mM dithiothreitol (DTT), pH 7.0) at a concentration of 5 mg/mL and further adding 4 μM of ThT is used.

In addition, an average value obtained by performing three times of measurement is used as a measured value. Measuring instrument: JASCO FP-8200 (manufactured by JASCO Corporation), measurement range: 440 to 600 nm, excitation wavelength: 450 nm, scan speed: medium, number of times of measurement: three times

In the measurement of the fluorescence intensity, a plate reader (for example, SYNERGY HTX (BIOTEC Co., Ltd.)) can be used. The plate reader can follow a temporal change in fluorescence intensity. The measurement may be performed according to the manual attached to the device.

In the present embodiment, it is preferable that a peak in a fluorescence intensity spectrum obtained by the fluorescence intensity measurement by ThT is within a range of 480 to 500 nm. In this case, the structural protein microbody has a β-sheet structure, and such a structural protein microbody is particularly likely to function as a core for forming a protein nanofiber.

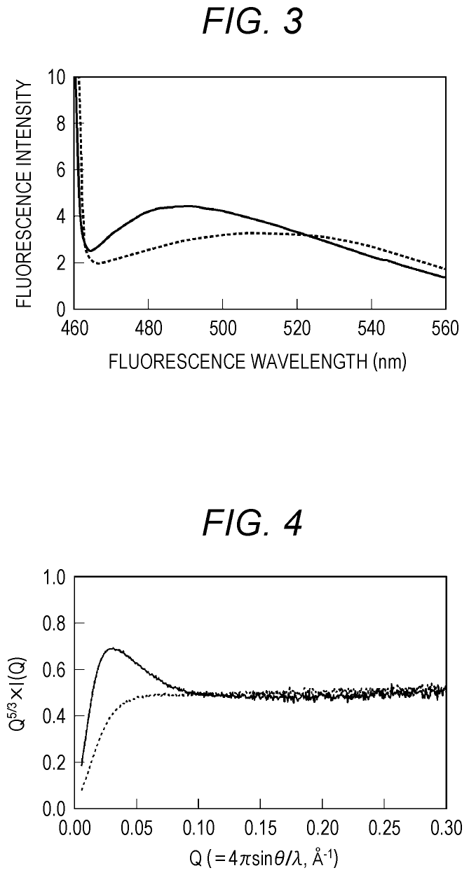

FIG. 3 is a diagram illustrating an example of a result of the fluorescence intensity measurement of the structural protein microbody by ThT staining. A1 of FIG. 3 (a graph indicated by the solid line of FIG. 3) is a measurement result obtained using a measurement sample obtained by dispersing the structural protein microbody in a first dispersion (aqueous solution of 6 M urea, 10 mM TrisHCl, and 5 mM DTT, pH 7.0) at a concentration of 5 mg/mL. A1 of FIG. 3 has a peak within a range of 480 to 500 nm, and it is confirmed from this that the structural protein microbody has a β-sheet structure.

X1 in FIG. 3 (a graph indicated by the dashed line in FIG. 3) is a measurement result obtained using a measurement sample obtained by dispersing the structural protein microbody in a second dispersion (aqueous solution of 5 M guanidine thiocyanate (GdmSCN), 10 mM trisHCl, and 5 mM DTT, pH 7.0) at a concentration of 5 mg/mL and then replacing the second dispersion with the first dispersion by dialysis. According to the findings by the present inventors, the structural protein microbody can maintain the structure thereof in the first dispersion, but does not maintain the structure thereof in the second dispersion, and is dissolved to act as a non-aggregated random coil structural protein molecule. Therefore, in X1 of FIG. 3, a peak is not present within the range of 480 to 500 nm. It can be seen from this result that the structural protein microbody having a β-sheet structure is not present in the second dispersion.

<(ii) Modified Kratky Plot of Small Angle X-Ray Scattering (SAXS)>

Specifically, the SAXS measurement can be performed under the following conditions. A measurement sample obtained by dispersing the structural protein microbody in a dispersion (aqueous solution of 6 M urea, 10 mM TrisHCl, and 5 mM DTT, pH 7.0) at a concentration of 5 mg/mL is used. Measuring apparatus: X-ray small angle scattering measuring apparatus NANO-Viewer (manufactured by Rigaku Corporation), X-ray generator MicroMAX007 (manufactured by Rigaku Corporation), detector PILATUS 200K (manufactured by DECTRIS Ltd.), measurement conditions: X-ray wavelength of 1.5418 Å (CuKα), room temperature (20° C.), exposure time of 30 minutes

After the measurement is performed under the above conditions, circumferential averaging is performed to obtain a one-dimensional profile. A modified Kratky plot can be obtained by analyzing the one-dimensional profile using IgorPro software (manufactured by WaveMetrics Inc.). In the present specification, the modified Kratky plot indicates a graph in which a horizontal axis is Q (=4π sin θ/λ) (unit: Å⁻¹) and a vertical axis is I(Q)×Q^{5/3} (unit: dimensionless).

In the present embodiment, it is considered that the characteristic of “a peak is present in a region where Q is 0.15 or less” indicates that the structural protein microbody has a spherical core portion having a high electron density. It is considered from this that the structural protein microbody satisfying (ii) has a core portion having a high electron density. Such a structural protein microbody is particularly likely to function as a core for forming a protein nanofiber.

In the present embodiment, in the modified Kratky plot of the structural protein microbody, a change width in a region where Q is 0.15 or more and 0.3 or less is preferably ±10% or less. Such a characteristic is considered to indicate that the structural protein microbody has a self-avoiding random walk chain. That is, it is considered that the structural protein microbody having both (ii) and this characteristic has a core portion having a high electron density and a random coil disposed to surround the core portion. Such a structural protein microbody is particularly likely to function as a core for forming a protein nanofiber.

In the present embodiment, a magnitude of the peak in the modified Kratky plot of small angle X-ray scattering (SAXS) is preferably 1.1 times or more and more preferably 1.15 times or more greater than an average value in a region where Q is 0.15 or more and 0.3 or less. In addition, the magnitude of the peak may be, for example, 2 times or less greater than the average value in the region where Q is 0.15 or more and 0.3 or less.

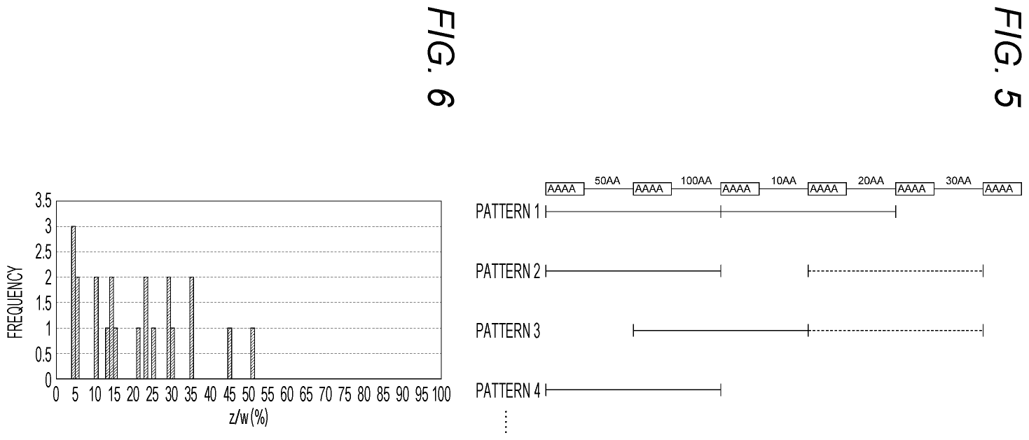

FIG. 4 is a diagram illustrating an example of the modified Kratky plot of the structural protein microbody. A2 of FIG. 4 (a graph indicated by the solid line of FIG. 4) is a measurement result obtained using a measurement sample obtained by dispersing the structural protein microbody in a first dispersion (aqueous solution of 6 M urea, 10 mM TrisHCl, and 5 mM DTT, pH 7.0) at a concentration of 5 mg/mL. A2 of FIG. 4 has a peak in a region where Q is 0.15 or less. In addition, a change width in a region where Q is 0.15 or more and 0.3 or less is +10% or less. It is confirmed from this result that the structural protein microbody has a core portion having a high electron density and a random coil disposed to surround the core portion.

X2 of FIG. 4 (a graph indicated by the dashed line of FIG. 4) is a measurement result obtained using a measurement sample obtained by dispersing the structural protein microbody in a second dispersion (aqueous solution of 5 M guanidine thiocyanate (GdmSCN), 10 mM trisHCl, and 5 mM DTT, pH 7.0) at a concentration of 5 mg/mL and then replacing the second dispersion with the first dispersion by dialysis. According to the findings by the present inventors, the structural protein microbody can maintain the structure thereof in the first dispersion, but does not maintain the structure thereof in the second dispersion, and is dissolved to act as a non-aggregated random coil structural protein molecule. Therefore, in X2 of FIG. 4, a peak is not present in the region where Q is 0.15 or less. It can be seen that the structural protein microbody having a core portion having a high electron density is not present.

<(iii) Aggregate of Structural Protein Molecules>

The structural protein microbody of the present embodiment is preferably an aggregate formed by aggregating two or more structural protein molecules. The number of aggregated structural protein molecules in the structural protein microbody is preferably 2 to 10, more preferably 2 to 5, and still more preferably 3.

The number of aggregated structural protein molecules in the structural protein microbody can be confirmed, for example, by comparing a molecular weight of non-aggregated structural protein molecules with a molecular weight of the aggregate. Specifically, the number of aggregated structural protein molecules in the structural protein microbody can be confirmed, for example, by an origin scattering intensity normalized by a weight concentration obtained by Guinier analysis. Since it is known that the origin scattering intensity is proportional to the molecular weight of the measurement sample, for example, when the origin scattering intensity of the structural protein microbody is 2.5 times or more and less than 3.5 times the origin scattering intensity of the non-aggregated structural protein molecules, the number of aggregated structural protein molecules in the structural protein microbody is 3.

That is, in the present embodiment, the origin scattering intensity of the structural protein microbody obtained by Guinier analysis is preferably 1.5 times or more, preferably 1.5 times or more and less than 10.5 times, and more preferably 1.5 or more times and less than 5.5 times the origin scattering intensity of the non-aggregated structural protein molecules, and may be 2.5 times or more and less than 3.5 times the origin scattering intensity of the non-aggregated structural protein molecules.

Specifically, the Guinier analysis can be performed by the following methods. First, as a first measurement sample group, measurement samples are prepared by dispersing structural protein microbodies in first dispersions (aqueous solution of 6 M urea, 10 mM TrisHCl, and 5 mM DTT, pH 7.0) at concentrations of 2 mg/mL, 4 mg/mL, 6 mg/mL, 8 mg/mL, and 10 mg/mL, respectively. Next, as a second measurement sample group, measurement samples were prepared by dispersing structural protein microbodies in second dispersions (aqueous solution of 5 M guanidine thiocyanate (GdmSCN), 10 mM trisHCl, and 5 mM DTT, pH 7.0) and then replacing the second dispersions with the first dispersions by dialysis at concentrations of 2 mg/mL, 4 mg/mL, 6 mg/mL, 8 mg/mL, and 10 mg/mL, respectively. SAXS measurement is performed on each of the first measurement sample group and the second measurement sample group by the following measuring apparatus under the following measuring conditions. Measuring apparatus: X-ray small angle scattering measuring apparatus NANO-Viewer (manufactured by Rigaku Corporation), X-ray generator MicroMAX007 (manufactured by Rigaku Corporation), detector PILATUS 200K (manufactured by DECTRIS Ltd.), measurement conditions: X-ray wavelength of 1.5418 Å (CuKα), room temperature (20° C.) exposure time of 30 minutes

A scattering curve of each of the samples obtained by the SAXS measurement is subjected to Guinier analysis, and from the result, an origin scattering intensity (1(0)) normalized at a concentration of 0 mg/ml is determined. The number of aggregated structural protein molecules in the structural protein microbody can be confirmed by comparing the origin scattering intensity normalized at the concentration determined from the first measurement sample group with the origin scattering intensity normalized at the concentration determined from the second measurement sample group. The origin scattering intensity normalized at the concentration determined from the first measurement sample group corresponds to the molecular weight of the structural protein microbody, and the origin scattering intensity normalized at the concentration determined from the second measurement sample group corresponds to the molecular weight of the non-aggregated structural protein molecules.

According to the findings by the present inventors, the structural protein microbody can maintain the structure thereof in the first dispersion, but does not maintain the structure thereof in the second dispersion, and is dissolved to act as a non-aggregated random coil structural protein molecule. Therefore, the number of aggregated structural protein molecules in the structural protein microbody can be confirmed by the above method.

Hereinafter, the X-ray small angle scattering (SAXS), the Guinier analysis, and the modified Kratky plot will be described in detail.

SAXS Measurement

A SAXS measurement is a method capable of evaluating a structure of a substance by measuring an X-ray that appears on a small angle side of 2θ<10° or less among X-rays scattered by irradiating the substance with X-rays.

FIG. 2 is a view for describing a measurement principle of the SAXS measurement. When scattering from one object is considered, how the X-ray scattered at the point A and the X-ray scattered at the point B illustrated in FIG. 2 strengthen each other in a scattering angle (angle between an incident direction and a scattering direction) 2θ direction is determined by a relationship between a difference in optical path length and a wavelength. A phase difference due to the optical path length is represented by r·Q₀−r·Q₁, in which Q₁ is an incident vector and Q₁ is a vector in the scattering angle 2θ direction. In the case of elastic scattering, since a wavelength is invariant,

$$\vert {Q}_{0} \vert = \vert {Q}_{1} \vert = \frac{2 π}{λ} \qquad [ Math . 1 ]$$

is obtained. When the scattering vector is defined as

$$\begin{matrix}Q = {Q}_{1} - {Q}_{0} , & [ Math . 2 ] \\ Q = \vert Q \vert = \frac{4 π sin θ}{λ} & [ Math . 3 ]\end{matrix}$$

is obtained, and a phase difference between two waves is rQ. Since the protein solution has a higher electron density than that of water, when scattering of water is subtracted from scattering of the protein solution, scattering due only to the protein structure can be obtained. In the case of solution scattering, scattering is concentric because the protein is isotropically present in the solution.

When a difference between a scattering amplitude F₁(Q) from one object and an electron density of water at an r point in the object is Δρ(r),

$${F}_{1} ( Q ) = \int Δρ ( r ) {e}^{- ir \cdot Q} {d}^{3} r \qquad [ Math . 4 ]$$

is obtained. That is, the scattering amplitude is a Fourier transform of the electron density. Since particles are irregularly present in the solution, scattering from individual particles averaged for all orientations is observed as an isotropic scattering intensity. When i₁(Q) represents a scattering intensity from the individual particles and I(Q) represents scattering from N particles,

$$\begin{matrix}{i}_{1} ( Q ) = {〈 {i}_{1} ( Q ) 〉}_{time} = 〈 {F}_{1} ( Q ) \cdot {F}_{1}^{*} ( Q ) 〉 \\ and & [ Math . 5 ] \\ I ( Q ) = {〈 {i}_{1} ( Q ) 〉}_{Ensemble} = {〈 F ( Q ) \cdot {F}^{*} ( Q ) 〉}_{Ensemble} & [ Math . 6 ]\end{matrix}$$

are obtained. In an ideal monodisperse system, scattering from N protein molecules is N times the scattering intensity from one spatially averaged molecule.

$$I ( Q ) = {Ni}_{1} ( Q ) \qquad [ Math . 7 ]$$

Since a product FT[φ(r)]·FT[Ψ(r)] of a Fourier transform FT[φ(r)] of φ(r) and a Fourier transform FT[Ψ(r)] of Ψ(r) is equal to a Fourier transform FT[φ(r)*Ψ(r)] of a convolution φ(r)*Ψ(r) of φ(r) and Ψ(r) (convolution theorem), the scattering intensity i₁(Q) of one molecule can be represented as follows.

$${i}_{1} ( Q ) = 〈 FT [ Δρ ( r ) ] FT [ Δρ ( - r ) ] 〉 = 〈 FT [ Δρ ( r ) * Δρ ( - r ) ] 〉 \qquad [ Math . 8 ]$$

Here, when an autocorrelation function γ(r)

$$γ ( r ) = Δρ ( r ) * Δρ ( - r ) = {\int }_{u} Δρ ( r + u ) * Δρ ( u ) {d}^{3} u \qquad [ Math . 9 ]$$

is introduced,

$${i}_{1} ( Q ) = 〈 FT [ γ ( r ) ] 〉 = 〈 {\int }_{0}^{∞} γ ( r ) {e}^{ir \cdot Q} {d}^{3} r 〉 \qquad [ Math . 10 ]$$

is obtained and further represented by

$$\begin{matrix}{i}_{1} ( Q ) = 4 π {\int }_{0}^{∞} {r}^{2} γ ( r ) \frac{sin ( rQ )}{rQ} dr = 4 π {\int }_{0}^{∞} P ( r ) \frac{sin ( rQ )}{rQ} dr ( A \text{-} 1 ) \\ and & [ Math . 11 ] \\ P ( r ) = {\int }_{0}^{∞} {r}^{2} γ ( r ) dr = {\int }_{0}^{∞} {r}^{2} Δρ ( r ) * Δρ ( - r ) dr . & [ Math . 12 ]\end{matrix}$$

The P(r) function of Equation (A-1) refers to a radial distribution function.

When scattering from one protein molecule is considered as a sum of scattering of atoms constituting the protein,

$${i}_{1} ( Q ) = {\sum }_{i = 1}^{N} {\sum }_{j = 1}^{N} {f}_{i} ( Q ) {f}_{j}^{*} ( Q ) {e}^{- i ( {r}_{i} - {r}_{j} ) {Q}_{i}} \qquad [ Math . 13 ]$$

is obtained, wherein fᵢ(Q) is scattering from the i-th atom and Debye is spatially averaged scattering, and

$${i}_{1} ( Q ) = {\sum }_{i = 1}^{N} {\sum }_{j = 1}^{N} {f}_{i} ( Q ) {f}_{j}^{*} ( Q ) \frac{sin ( {r}_{ij} Q )}{{r}_{ij} Q} , \\ {r}_{ij} = \vert {r}_{i} - {r}_{j} \vert \qquad [ Math . 14 ]$$

is obtained. When a continuous electron density is considered, it can be represented by

$${i}_{1} ( Q ) = {\int }_{{r}_{1}} {\int }_{{r}_{2}} Δρ ( {r}_{1} ) Δρ ( {r}_{2} ) \frac{sin ( {r}_{12} Q )}{{r}_{12} Q} {d}^{3} {r}_{1} {d}^{3} {r}_{2} ( A \text{-} 2 ) . \qquad [ Math . 15 ]$$

Guinier Analysis

A Guinier plot can be linearly approximated in a small angle region by plotting a logarithm of the scattering intensity against a square of the scattering vector. In Equation (A-1), a region where a scattering angle is significantly small is considered.

When Taylor expansion is performed like

$$\frac{sin ( rQ )}{rQ} = 1 - \frac{{( rQ )}^{2}}{3 !} + \frac{{( rQ )}^{4}}{5 !} + ⋯ , \qquad [ Math . 16 ]$$

it is approximated to

$$\begin{matrix}I ( Q ) ≅ I ( 0 ) [ 1 - {kQ}^{2} ] ≅ I ( 0 ) {e}^{- {kQ}^{2}} . \\ Here , & [ Math . 17 ] \\ I ( 0 ) = 4 π {\int }_{0}^{∞} P ( r ) dr , \\ k = \frac{1}{6} \frac{{\int }_{0}^{∞} {r}^{2} P ( r ) dr}{{\int }_{0}^{∞} P ( r ) dr} & [ Math . 18 ]\end{matrix}$$

is obtained.

Similarly, when Taylor expansion with respect to I(Q) extended to scattering from all the structural protein molecules in the solution is performed on Equation (A-2),

$$\begin{matrix}I ( Q ) = & {\int }_{{r}_{1}} {\int }_{{r}_{2}} Δρ ( {r}_{1} ) Δρ ( {r}_{2} ) \frac{sin ( {r}_{12} Q )}{{r}_{12} Q} {d}^{3} {r}_{1} {d}^{3} {r}_{2} \\ = & {\int }_{{r}_{1}} {\int }_{{r}_{2}} Δρ ( {r}_{1} ) Δρ ( {r}_{2} ) {d}^{3} {r}_{1} {d}^{3} {r}_{2} - \\ & \frac{{Q}^{2}}{6} {\int }_{{r}_{1}} {\int }_{{r}_{2}} Δρ ( {r}_{1} ) Δρ ( {r}_{2} ) {r}_{12}^{2} {d}^{2} {r}_{1} {d}^{3} {r}_{2}\end{matrix} \qquad [ Math . 19 ]$$

is obtained, and in this case,

$$\begin{matrix}[ Math . 20 ] & \\ k = \frac{1}{6} \frac{{\int }_{{r}_{1}} {\int }_{{r}_{2}} Δρ ( {r}_{1} ) Δρ ( {r}_{2} ) {{r}_{12}}^{2} {d}^{3} {r}_{1} {d}^{3} {r}_{2}}{{\int }_{{r}_{1}} {\int }_{{r}_{2}} Δρ ( {r}_{1} ) Δρ ( {r}_{2} ) {d}^{3} {r}_{1} {d}^{3} {r}_{2}} & \end{matrix}$$

is obtained.

When the coordinates are exchanged and r₀ is set as the origin, the equation can be calculated as

$$\begin{matrix}[ Math . 21 ] & \\ k = \frac{1}{6} [ \frac{2 {\int }_{r} Δ ρ ( r - {r}_{0} ) {\vert r - {r}_{0} \vert}^{2} {d}^{3} r}{{\int }_{{r}_{1}} Δρ ( r ) {d}^{3} r} - 2 {\{ \frac{{\int }_{r} Δρ ( r - {r}_{0} ) ( r - {r}_{0} ) {d}^{3} r}{{\int }_{{r}_{1}} Δρ ( r ) {d}^{3} r} \}}^{2} ] . & \end{matrix}$$

Since r₀ can coincide with the gravity center of the molecule, the second term of the above equation becomes zero, and

$$\begin{matrix}[ Math . 22 ] & \\ k = \frac{1}{3} \frac{{\int }_{r} Δρ ( r ) {r}^{2} {d}^{3} r}{{\int }_{r} Δρ ( r ) {d}^{3} r} = \frac{1}{3} R {g}^{2} & \end{matrix}$$

is obtained. Accordingly,

$$\begin{matrix}[ Math . 23 ] & \\ I ( Q ) = I ( 0 ) exp [ - \frac{1}{3} R {g}^{2} {Q}^{2} ] & \text{(A-3)}\end{matrix}$$

is obtained.

This is called Guinier's law. A radius of rotation Rg² is defined by Equation (A-3) as follows.

$$\begin{matrix}[ Math . 24 ] & \\ R {g}^{2} = \frac{{\int }_{r} Δρ ( r ) {r}^{2} {d}^{3} r}{{\int }_{r} Δρ ( r ) {d}^{3} r} & \end{matrix}$$

By taking natural logarithms of both sides of Equation (A-3),

$$\begin{matrix}[ Math . 25 ] & \\ ln [ I ( Q ) ] = ln [ I ( 0 ) ] - \frac{1}{3} R {g}^{2} {Q}^{2} & \end{matrix}$$

is obtained.

A plot in which Q² is plotted on the horizontal axis and ln[I(Q)] is plotted on the vertical axis is referred to as a Guinier plot. A linear region exists in the small angle region of the scattering curve, and the origin scattering intensity I(0) is determined from the Y-intercept obtained by extrapolating a straight line of a radius of inertia Rg² from the slope thereof to the origin. In Equation (A-1), Q=0, that is, the scattering intensity at the origin is represented by

$$\begin{matrix}[ Math . 26 ] & \\ {i}_{1} ( 0 ) = {\int }_{r} {\int }_{r '} Δρ ( r ) Δρ ( {r}^{′} ) {d}^{3} r {d}^{3} {r}^{′} & \end{matrix}$$

$$\begin{matrix}[ Math . 27 ] & \\ {i}_{1} ( 0 ) = Δ {m}^{2} = {( m - {m}_{0} )}^{2} = {[ \frac{M}{{N}_{A}} {v}_{p} ( ρ - {ρ}_{0} ) ]}^{2} & \end{matrix}$$

is obtained because it is an equation related to a total number of protein electrons having an electron density higher than that of water. Here, each of m and m₀ is the number of electrons of the protein and water in a volume occupied by one protein molecule, M and νₚ are a molecular weight and a partial volume of the protein, respectively, and ρ and ρ₀ are electron densities of the protein and water, respectively. In scattering from a system in which N structural protein molecules are present in an irradiation volume V (ml) of X-rays,

$$\begin{matrix}[ Math . 29 ] \\ I ( 0 ) = N {i}_{1} ( 0 ) = {\frac{c M V}{{N}_{A}} [ {v}_{p} ( ρ - {ρ}_{0} ) ]}^{ 2}\end{matrix}$$

is obtained using a protein concentration c of the following equation:

$$\begin{matrix}[ Math . 28 ] \\ c = \frac{N M}{{N}_{A}} V ( \text{mg/ml)}\end{matrix}$$

An apparent molecular weight of the structural protein molecule can be estimated by normalizing and comparing origin scattering intensities of a standard sample and a target protein by a concentration. From this, it is possible to determine the number of aggregated structural proteins in the solution.

Kratky Plot

A Kratky plot is obtained by plotting I(Q)·Q² obtained by multiplying the scattering intensity I(Q) by Q² against Q. The scattering curve obtained from a compact spherical structure has a region where I(Q) follows Q⁻⁴ (Porod side), that is, there is a region where I(Q)·Q² follows Q⁻², and a peak always appears in the Kratky plot. A peak present on a smaller angle side indicates a larger radius of inertia, and a peak present on a more middle angle region side indicates a smaller radius of inertia. In addition, the scattering curve from an ideal random coil (Gaussian chain) present in a good solvent follows Q⁻². Therefore, the Kratky plot asymptotically approaches a straight line parallel to the horizontal axis. A segment that can be regarded as a straight line called a persistence length is present in an actual random chain molecule. Therefore, the scattering curve on a wide angle side is proportional to the scattering curve I(Q)∝Q⁻¹ from a needle-shaped molecule, and the Kratky plot asymptotically approaches the straight line passing through the origin. The sphericity and compactness of the protein can be observed by comparing the Kratky plots of the protein. The modified Kratky plot is a Kratky plot obtained by considering a self-avoiding random walk chain. In the case of the self-avoiding random walk chain, a wide angle region is proportional to Q^{-5/3}. For example, in a modified Kratky plot of a polymer having a structure in which a core portion having a high electron density, such as a dendrimer or a star polymer, and a random coil surrounding the core portion are arranged, a peak is observed in a small angle region, and a horizontal region is observed in a wide angle region.

An average particle size of the structural protein microbodies according to the present embodiment is preferably 1 to 50 nm, more preferably 3 to 30 nm, and still more preferably 9 to 15 nm.

In the present specification, the average particle size of the structural protein microbodies indicates a volume average size measured by a dynamic scattering method. More specifically, the average particle size of the structural protein microbodies is measured by the following method.

First, as a measurement sample group, measurement samples are prepared by dispersing structural protein microbodies in first dispersions (aqueous solution of 6 M urea, 10 mM TrisHCl, and 5 mM DTT, pH 7.0) at concentrations of 2 mg/mL, 4 mg/mL, 6 mg/mL, 8 mg/mL, and 10 mg/mL, respectively. Next, a particle size distribution of each of the measurement samples is measured by a dynamic light scattering method under the following conditions to determine a volume average size. Measuring apparatus: ZETASIZER nano-ZS (manufactured by Malvern Panalytical), measurement temperature: 20° C.

The measurement is performed 5 times for each measurement sample to determine an average value of the obtained measured values. From the concentration and the measured value (average value) of each of the measurement samples, a plot of the average particle size against the concentration is obtained, and 0 concentration extrapolation excluding an intermolecular interaction is performed. The value obtained by the 0 concentration extrapolation is defined as an average particle size of the structural protein microbodies.

The structural protein constituting the structural protein microbody will be described in detail below.

Examples of the structural protein can include a natural structural protein and a modified structural protein (an artificial structural protein). In addition, an example of the modified structural protein can include any structural protein that can be produced on an industrial scale. Specific examples of the structural protein can include spider silk, silkworm moth silk, psychidae silk, hornet silk, keratin, collagen, elastin, resilin, and proteins derived therefrom.

As the structural protein constituting the structural protein microbody, a fibroin-like protein (hereinafter, simply referred to as “fibroin”) is preferable, modified fibroin is more preferable, and modified spider silk fibroin is still more preferable.

(Modified Fibroin)

The modified fibroin according to the present embodiment is a protein containing a domain sequence represented by Formula 1: [(A)ₙ motif-REP]ₘ or Formula 2: [(A)ₙ motif-REP]ₘ−(A)ₙ motif. An amino acid sequence (N-terminal sequence and C-terminal sequence) may be further added to either or both of the N-terminal side and the C-terminal side of the domain sequence of the modified fibroin. The N-terminal sequence and the C-terminal sequence are not limited thereto, but, typically are regions having no repetitions of amino acid motifs characterized in fibroin, and each consist of amino acids of approximately 100 residues.

The term “modified fibroin” in the present specification refers to artificially produced fibroin (artificial fibroin). The modified fibroin may be fibroin in which a domain sequence is different from an amino acid sequence of naturally derived fibroin or may be fibroin in which a domain sequence is the same as an amino acid sequence of naturally derived fibroin. The “naturally derived fibroin” referred to in the present specification is also a protein containing a domain sequence represented by Formula 1: [(A)ₙ motif-REP]ₘ or Formula 2: [(A)ₙ motif-REP]ₘ−(A)ₙ motif.

The “modified fibroin” may be fibroin obtained by using an amino acid sequence of naturally derived fibroin as it is, fibroin in which an amino acid sequence is modified based on an amino acid sequence of naturally derived fibroin (for example, fibroin in which an amino acid sequence is modified by modifying a cloned gene sequence of naturally derived fibroin), or fibroin artificially designed and synthesized independently of naturally derived fibroin (for example, fibroin having a desired amino acid sequence by chemically synthesizing a nucleic acid encoding a designed amino acid sequence).

In the present specification, the term “domain sequence” refers to an amino acid sequence which produces a crystalline region (typically, corresponding to an (A)ₙ motif of an amino acid sequence) and an amorphous region (typically, corresponding to REP of an amino acid sequence) specific to fibroin, and refers to an amino acid sequence represented by Formula 1: [(A)ₙ motif-REP]ₘ or Formula 2: [(A)ₙ—motif-REP]ₘ−(A)ₙ motif. Here, the (A)ₙ motif represents an amino acid sequence mainly consisting of alanine residues, and the number of amino acid residues is 2 to 27. The number of amino acid residues in the (A)ₙ motif may be an integer of 2 to 20, 4 to 27, 4 to 20, 8 to 20, 10 to 20, 4 to 16, 8 to 16, or 10 to 16. In addition, a proportion of the number of alanine residues to a total number of amino acid residues in the (A)ₙ motif may be 40% or more, and may also be 60% or more, 70% or more, 80% or more, 83% or more, 85% or more, 86% or more, 90% or more, 95% or more, or 100% (which means that the (A)ₙ motif consists of only alanine residues). At least a plurality of seven (A)ₙ motifs present in the domain sequence may consist of only alanine residues. The REP represents an amino acid sequence consisting of 2 to 200 amino acid residues. The REP may be an amino acid sequence consisting of 10 to 200 amino acid residues. m represents an integer of 2 to 300, and may be an integer of 10 to 300. A plurality of (A)ₙ motifs may be the same amino acid sequences or different amino acid sequences. A plurality of REPs may be the same amino acid sequences or different amino acid sequences.

The modified fibroin according to the present embodiment can be obtained by, for example, performing modification of an amino acid sequence corresponding to substitution, deletion, insertion, and/or addition of one or a plurality of amino acid residues with respect to a cloned gene sequence of naturally derived fibroin. Substitution, deletion, insertion, and/or addition of the amino acid residues can be performed by methods well known to those skilled in the art, such as site-directed mutagenesis. Specifically, the modification may be performed according to a method described in literatures such as Nucleic Acid Res. 10, 6487 (1982), and Methods in Enzymology, 100, 448 (1983).

The naturally derived fibroin is a protein containing a domain sequence represented by Formula 1: [(A)ₙ motif-REP]ₘ or Formula 2: [(A)ₙ motif-REP]ₘ−(A)ₙ motif, and a specific example thereof can include fibroin produced by insects or spiders. Examples of the fibroin produced by insects can include silk proteins produced by silkworms such as Bombyx mori, Bombyx mandarina, Antheraea yamamai, Anteraea pernyi, Eriogyna pyretorum, Pilosamia Cynthia ricini, Samia cynthia, Caligura japonica, Antheraea mylitta, and Antheraea assama and a hornet silk protein secreted by larvae of Vespa simillima xanthoptera.

A more specific example of the fibroin produced by insects can include a silkworm fibroin L chain (GenBank Accession No. M76430 (base sequence) and AAA27840.1 (amino acid sequence)).

Examples of the fibroin produced by spiders can include spider silk proteins produced by spiders belonging to the order Araneae. Specific examples thereof can include spider silk proteins produced by spiders belonging to the genus Araneus, such as Araneus ventricosus, Araneus diadematus, Araneus pinguis, Araneus pentagrammicus, and Araneus nojimai, spiders belonging to the genus Neoscona, such as Neoscona scylla, Neoscona nautica, Neoscona adianta, and Neoscona scylloides, spiders belonging to the genus Pronus, such as Pronous minutus, spiders belonging to the genus Cyrtarachne, such as Cyrtarachne bufo and Cyrtarachne inaequalis, spiders belonging to the genus Gasteracantha, such as Gasteracantha kuhlii and Gasteracantha mammosa, spiders belonging to the genus Ordgarius, such as Ordgarius hobsoni and Ordgarius sexspinosus, spiders belonging to the genus Argiope, such as Argiope amoena, Argiope minuta, and Argiope bruennichi, spiders belonging to the genus Arachnura, such as Arachnura logio, spiders belonging to the genus Acusilas, such as Acusilas coccineus, spiders belonging to the genus Cytophora, such as Cyrtophora moluccensis, Cyrtophora exanthematica, and Cyrtophora unicolor, spiders belonging to the genus Poltys, such as Poltys illepidus, spiders belonging to the genus Cyclosa, such as Cyclosa octotuberculata, Cyclosa sedeculata, Cyclosa vallata, and Cyclosa atrata, and spiders belonging to the genus Chorizopes, such as Chorizopes nipponicus, and spider silk proteins produced by spiders belonging to the family Tetragnathidae, such as spiders belonging to the genus Tetragnatha, such as Tetragnatha praedonia, Tetragnatha maxillosa, Tetragnatha extensa, and Tetragnatha squamata, spiders belonging to the genus Leucauge, such as Leucauge magnifica, Leucauge blanda, and Leucauge subblanda, spiders belonging to the genus Nephila, such as Nephila clavata and Nephila pilipes, spiders belonging to the genus Menosira, such as Menosira ornata, spiders belonging to the genus Dyschiriognatha, such as Dyschiriognatha tenera, spiders belonging to the genus Latrodectus, such as Latrodectus mactans, Latrodectus hasseltii, Latrodectus geometricus, and Latrodectus tredecimguttatus, and spiders belonging to the genus Euprosthenops. Examples of the spider silk proteins can include dragline silk proteins such as MaSps (MaSp1 and MaSp2) and ADFs (ADF3 and ADF4), MiSps (MiSp1 and MiSp2), AcSp, PySp, and Flag.

More specific examples of the spider silk protein produced by spiders can include fibroin-3 (adf-3) [derived from Araneus diadematus] (GenBank Accession No. AAC47010 (amino acid sequence), U47855 (base sequence)), fibroin-4 (adf-4) [derived from Araneus diadematus] (GenBank Accession No. AAC47011 (amino acid sequence), U47856 (base sequence)), dragline silk protein spidroin 1 [derived from Nephila clavipes] (GenBank Accession No. AAC04504 (amino acid sequence), U37520 (base sequence)), major ampullate spidroin 1 [derived from Latrodectus hesperus] (GenBank Accession No. ABR68856 (amino acid sequence), EF595246 (base sequence)), dragline silk protein spidroin 2 [derived from Nephila clavata] (GenBank Accession No. AAL32472 (amino acid sequence), AF441245 (base sequence)), major ampullate spidroin 1 [derived from Euprosthenops australis] (GenBank Accession No. CAJ00428 (amino acid sequence), AJ973155 (base sequence)), and major ampullate spidroin 2 [Euprosthenops australis] (GenBank Accession No. CAM32249.1 (amino acid sequence), AM490169 (base sequence)), minor ampullate silk protein 1 [Nephila clavipes] (GenBank Accession No. AAC14589.1 (amino acid sequence)), minor ampullate silk protein 2 [Nephila clavipes] (GenBank Accession No. AAC14591.1 (amino acid sequence)), and minor ampullate spidroin-like protein [Nephilengys cruentata] (GenBank Accession No. ABR37278.1 (amino acid sequence).

A more specific example of the naturally derived fibroin can include fibroin with sequence information registered in NCBI GenBank. For example, sequences thereof can be confirmed by extracting sequences in which spidroin, ampullate, fibroin, “silk and polypeptide”, or “silk and protein” is described as a keyword in DEFINITION among sequences containing INV as DIVISION among sequence information registered in NCBI GenBank, sequences in which a specific character string of products is described from CDS, or sequences in which a specific character string is described from SOURCE to TISSUE TYPE.

The modified fibroin according to the present embodiment may be modified silk fibroin (in which an amino acid sequence of silk protein produced by silkworm is modified), or may be modified spider silk fibroin (in which an amino acid sequence of a spider silk protein produced by spiders is modified). As the modified fibroin, modified spider silk fibroin is preferred because it is more excellent in flame retardancy.

Specific examples of the modified fibroin can include modified fibroin derived from a major dragline silk protein produced in a major ampullate gland of a spider (first modified fibroin), modified fibroin containing a domain sequence in which a content of glycine residues is reduced (second modified fibroin), modified fibroin containing a domain sequence in which a content of an (A)ₙ motif is reduced (third modified fibroin), modified fibroin in which a content of glycine residues and a content of an (A)ₙ motif are reduced (fourth modified fibroin), modified fibroin containing a domain sequence including a region locally having a high hydropathy index (fifth modified fibroin), and modified fibroin containing a domain sequence in which a content of glutamine residues is reduced (sixth modified fibroin).

An example of the first modified fibroin can include a protein containing a domain sequence represented by Formula 1: [(A)ₙ motif-REP]ₘ. In the first modified fibroin, the number of amino acid residues in the (A)ₙ motif is preferably an integer of 3 to 20, more preferably an integer of 4 to 20, still more preferably an integer of 8 to 20, still more preferably an integer of 10 to 20, still more preferably an integer of 4 to 16, particularly preferably an integer of 8 to 16, and most preferably an integer of 10 to 16. In the first modified fibroin, the number of amino acid residues constituting REP in Formula 1 is preferably 10 to 200 residues, more preferably 10 to 150 residues, and still more preferably 20 to 100 residues, and still more preferably 20 to 75 residues. In the first modified fibroin, a total number of glycine residues, serine residues, and alanine residues contained in the amino acid sequence represented by Formula 1: [(A)ₙ motif-REP]ₘ is preferably 40% or more, more preferably 60% or more, and still more preferably 70% or more, with respect to a total number of amino acid residues.

The first modified fibroin may be a polypeptide having an amino acid sequence unit represented by Formula 1: [(A)ₙ motif-REP]ₘ, and having a C-terminal sequence which is an amino acid sequence set forth in any one of SEQ ID NOs: 1 to 3 or a C-terminal sequence which is an amino acid sequence having 90% or more homology with the amino acid sequence set forth in any one of SEQ ID NOs: 1 to 3.

The amino acid sequence set forth in SEQ ID NO: 1 is identical to an amino acid sequence consisting of 50 amino acid residues of the C-terminus of an amino acid sequence of ADF3 (GI: 1263287, NCBI). The amino acid sequence set forth in SEQ ID NO: 2 is identical to an amino acid sequence set forth in SEQ ID NO: 1 in which 20 amino acid residues have been removed from the C-terminus. The amino acid sequence set forth in SEQ ID NO: 3 is identical to an amino acid sequence set forth in SEQ ID NO: 1 in which 29 amino acid residues have been removed from the C-terminus.

A more specific example of the first modified fibroin can include modified fibroin having an amino acid sequence set forth in (1-i) SEQ ID NO: 4 (recombinant spider silk protein ADF3KaiLargeNRSH1), or (1-ii) an amino acid sequence having 90% or more sequence identity with the amino acid sequence set forth in (1-i) SEQ ID NO: 4. The sequence identity is preferably 95% or more.

The amino acid sequence set forth in SEQ ID NO: 4 is an amino acid sequence obtained by the following mutation: in an amino acid sequence of ADF3 in which an amino acid sequence (SEQ ID NO: 5) consisting of a start codon, a His 10-tag and an HRV3C protease (Human rhinovirus 3C protease) recognition site is added to the N-terminus, the 1st to 13th repetitive regions are about doubled and the translation ends at the 1,154th amino acid residue. The C-terminal amino acid sequence of the amino acid sequence set forth in SEQ ID NO: 4 is identical to the amino acid sequence set forth in SEQ ID NO: 3.

The modified fibroin of (1-i) may consist of the amino acid sequence set forth in SEQ ID NO: 4.

The domain sequence of the second modified fibroin has an amino acid sequence in which a content of glycine residues is reduced, as compared with the naturally derived fibroin. It can be said that the second modified fibroin has an amino acid sequence corresponding to an amino acid sequence in which at least one or a plurality of glycine residues in REP are substituted with another amino acid residue, as compared with the naturally derived fibroin.

The domain sequence of the second modified fibroin may have an amino acid sequence corresponding to an amino acid sequence in which one glycine residue in at least one or the plurality of motif sequences is substituted with another amino acid residue, in at least one motif sequence selected from GGX and GPGXX (where G represents a glycine residue, P represents a proline residue, and X represents an amino acid residue other than glycine) in REP, as compared with the naturally derived fibroin.

In the second modified fibroin, a proportion of the motif sequences in which the above-described glycine residue is substituted with another amino acid residue may be 10% or more with respect to the entire motif sequences.

The second modified fibroin may contain a domain sequence represented by Formula 1: [(A)ₙ motif-REP]ₘ and may have an amino acid sequence in which z/w is 30% or more, 40% or more, 50% or more, or 50.9% or more, in which a total number of amino acid residues in an amino acid sequence consisting of XGX (where X represents an amino acid residue other than glycine) contained in all REPs in a sequence excluding the sequence from the (A)ₙ motif located at the most C-terminal side to the C-terminus of the domain sequence from the domain sequence is z, and a total number of amino acid residues in a sequence excluding the sequence from the (A)ₙ motif located at the most C-terminal side to the C-terminus of the domain sequence from the domain sequence is w. The number of alanine residues with respect to the total number of amino acid residues in the (A)ₙ motif is 83% or more, preferably 86% or more, more preferably 90% or more, still more preferably 95% or more, and still more preferably 100% (which means that the (A)ₙ motif consists of only alanine residues).

The second modified fibroin is preferably one in which a content ratio of the amino acid sequence consisting of XGX is increased by substituting one glycine residue in the GGX motif with another amino acid residue. In the second modified fibroin, the content ratio of the amino acid sequence consisting of GGX in the domain sequence is preferably 30% or less, more preferably 20% or less, still more preferably 10% or less, even still more preferably 6% or less, still further preferably 4% or less, and particularly preferably 2% or less. The content ratio of the amino acid sequence consisting of GGX in the domain sequence can be calculated by the same method as the following calculation method of a content ratio (z/w) of the amino acid sequence consisting of XGX.

The calculation method of z/w will be described in more detail. First, the amino acid sequence consisting of XGX is extracted from all the REPs contained in the sequence excluding the sequence from the (A)ₙ motif located at the most C-terminal side to the C-terminus of the domain sequence from the domain sequence in the fibroin containing the domain sequence represented by Formula 1: [(A)ₙ motif-REP]ₘ (modified fibroin or naturally derived fibroin). A total number of amino acid residues consisting of XGX is z. For example, in a case where 50 amino acid sequences consisting of XGX are extracted (there is no overlap), z is 50×3=150. In addition, for example, in a case where X (central X) contained in two XGXs exists as in a case of the amino acid sequence consisting of XGXGX, z is calculated by subtracting the overlapping portion (in a case of XGXGX, it is 5 amino acid residues). w is a total number of amino acid residues contained in a sequence excluding the sequence from the (A)ₙ motif located at the most C-terminal side to the C-terminus of the domain sequence from the domain sequence. For example, in the case of the domain sequence illustrated in FIG. 5, w is 4+50+4+100+4+10+4+20+4+30=230 (excluding the (A)ₙ motif located at the most C-terminal side). Next, z/w (%) can be calculated by dividing z by w.

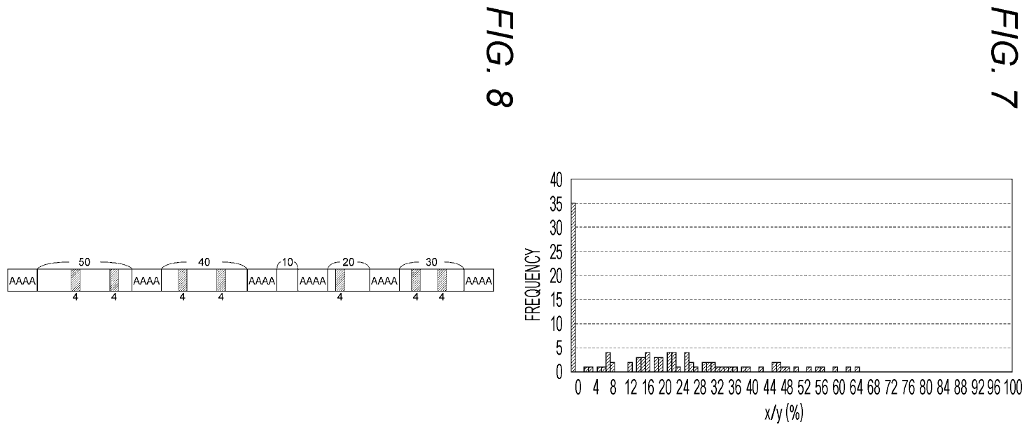

Here, z/w in the naturally derived fibroin will be described. First, as described above, 663 types of fibroins (415 types of fibroins derived from spiders among them) were extracted by confirming fibroins with amino acid sequence information registered in NCBI GenBank by an exemplified method. z/w was calculated by the above-described calculation method from the amino acid sequences of the naturally derived fibroins which contain a domain sequence represented by Formula 1: [(A)ₙ motif-REP]ₘ and in which the content ratio of the amino acid sequence consisting of GGX in the fibroin is 6% or less, among all the extracted fibroins. The results are illustrated in FIG. 6. In FIG. 6, the horizontal axis represents z/w (%), and the vertical axis represents a frequency. As is clear from FIG. 6, the values of z/w in the naturally derived fibroin are all smaller than 50.9% (the largest value is 50.86%).

In the second modified fibroin, z/w is preferably 50.9% or more, more preferably 56.1% or more, still more preferably 58.7% or more, even still more preferably 70% or more, and still further preferably 80% or more. An upper limit of z/w is not particularly limited, but may be, for example, 95% or less.

The second modified fibroin can be obtained by, for example, substituting and modifying at least a part of a base sequence encoding a glycine residue from a cloned gene sequence of naturally derived fibroin so as to encode another amino acid residue. In this case, one glycine residue in a GGX motif or a GPGXX motif may be selected as the glycine residue to be modified, and substitution may be performed so that z/w is 50.9% or more. In addition, the second modified fibroin can also be obtained by, for example, designing an amino acid sequence satisfying each of the above aspects from the amino acid sequence of the naturally derived fibroin, and chemically synthesizing a nucleic acid encoding the designed amino acid sequence. In any case, in addition to the modification corresponding to substitution of a glycine residue in the REP with another amino acid residue from the amino acid sequence of the naturally derived fibroin, modification of the amino acid sequence corresponding to substitution, deletion, insertion, and/or addition of one or a plurality of amino acid residues may be performed.

The above-described another amino acid residue is not particularly limited as long as it is an amino acid residue other than a glycine residue, but it is preferably a hydrophobic amino acid residue such as a valine (V) residue, a leucine (L) residue, an isoleucine (I) residue, a methionine (M) residue, a proline (P) residue, a phenylalanine (F) residue, or a tryptophan (W) residue, or a hydrophilic amino acid residue such as a glutamine (Q) residue, an asparagine (N) residue, a serine (S) residue, a lysine (K) residue, or a glutamic acid (E) residue, more preferably a valine (V) residue, a leucine (L) residue, an isoleucine (I) residue, a phenylalanine (F) residue, or a glutamine (Q) residue, and still more preferably a glutamine (Q) residue.

A more specific example of the second modified fibroin can include a modified fibroin having (2-i) an amino acid sequence set forth in SEQ ID NO: 6 (Met-PRT380), SEQ ID NO: 7 (Met-PRT410), SEQ ID NO: 8 (Met-PRT525), or SEQ ID NO: 9 (Met-PRT799), or (2-ii) an amino acid sequence having 90% or more sequence identity with the amino acid sequence set forth in SEQ ID NO: 6, SEQ ID NO: 7, SEQ ID NO: 8, or SEQ ID NO: 9.

The modified fibroin of (2-i) will be described. The amino acid sequence set forth in SEQ ID NO: 6 is obtained by substituting GQX for all GGXs in REP of the amino acid sequence set forth in SEQ ID NO: 10 (Met-PRT313) corresponding to the naturally derived fibroin. The amino acid sequence set forth in SEQ ID NO: 7 is obtained by deleting every other two (A)ₙ motifs from the N-terminal side to the C-terminal side from the amino acid sequence set forth in SEQ ID NO: 6 and further inserting one [(A)ₙmotif-REP] before the C-terminal sequence. The amino acid sequence set forth in SEQ ID NO: 8 is obtained by inserting two alanine residues at the C-terminal side of each (A)ₙ motif of the amino acid sequence set forth in SEQ ID NO: 7 and further substituting a part of glutamine (Q) residues with a serine (S) residue to delete a part of amino acids at the C-terminal side so as to be almost the same as a molecular weight of SEQ ID NO: 7. The amino acid sequence set forth in SEQ ID NO: 9 is an amino acid sequence obtained by adding a predetermined hinge sequence and a His tag sequence to the C-terminus of a sequence obtained by repeating a region of 20 domain sequences (where several amino acid residues on the C-terminal side of the region are substituted) present in the amino acid sequence set forth in SEQ ID NO: 7 four times.

A value of z/w in the amino acid sequence set forth in SEQ ID NO: 10 (corresponding to naturally derived fibroin) is 46.8%. The values of z/w in the amino acid sequence set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are 58.7%, 70.1%, 66.1%, and 70.0%, respectively. In addition, the values of x/y in the amino acid sequences set forth in SEQ ID NO: 10, SEQ ID NO: 6, SEQ ID NO: 7, SEQ ID NO: 8, and SEQ ID NO: 9 at a Giza ratio (described below) of 1:1.8 to 11.3 are 15.0%, 15.0%, 93.4%, 92.7%, and 89.8%, respectively.

The modified fibroin of (2-i) may consist of the amino acid sequence set forth in SEQ ID NO: 6, SEQ ID NO: 7, SEQ ID NO: 8, or SEQ ID NO: 9.

The modified fibroin of (2-ii) includes an amino acid sequence having a sequence identity of 90% or more with the amino acid sequence set forth in SEQ ID NO: 6, SEQ ID NO: 7, SEQ ID NO: 8, or SEQ ID NO: 9. The modified fibroin of (2-ii) is a protein containing the domain sequence represented by Formula 1: [(A)ₙ motif-REP]ₘ. The sequence identity is preferably 95% or more.

The modified fibroin of (2-ii) preferably has 90% or more sequence identity with the amino acid sequence set forth in SEQ ID NO: 6, SEQ ID NO: 7, SEQ ID NO: 8, or SEQ ID NO: 9, and z/w is preferably 50.9% or more, in which the total number of amino acid residues in the amino acid sequence consisting of XGX (where X represents the amino acid residue other than glycine) in the REP is z, and the total number of amino acid residues in the REP in the domain sequence is w.

The second modified fibroin may have a tag sequence at either or both of the N-terminus and C-terminus. This makes it possible to isolate, immobilize, detect, and visualize the modified fibroin.

An example of the tag sequence can include an affinity tag using specific affinity (binding property and affinity) with another molecule. A specific example of the affinity tag includes a histidine tag (His tag). The His tag is a short peptide in which about 4 to 10 histidine residues are arranged and has a property of specifically binding to a metal ion such as nickel. Thus, the His tag can be used for isolation of modified fibroin by chelating metal chromatography. A specific example of the tag sequence can include an amino acid sequence set forth in SEQ ID NO: 11 (amino acid sequence having a His tag sequence and a hinge sequence).

Also, a tag sequence such as glutathione-S-transferase (GST) that specifically binds to glutathione, and a maltose binding protein (MBP) that specifically binds to maltose can also be utilized.

Further, an “epitope tag” utilizing an antigen-antibody reaction can also be used. By adding a peptide (epitope) illustrating antigenicity as a tag sequence, an antibody against the epitope can be bound. Examples of the epitope tag include an HA (peptide sequence of hemagglutinin of influenza virus) tag, a myc tag, and a FLAG tag. The modified fibroin can easily be purified with high specificity by utilizing an epitope tag.

Further, it is also possible to use a tag sequence which can be cleaved with a specific protease. By treating a protein adsorbed through the tag sequence with protease, it is also possible to recover the modified fibroin cleaved from the tag sequence.

A more specific example of the modified fibroin having a tag sequence can include modified fibroin having (2-iii) an amino acid sequence set forth in SEQ ID NO: 12 (PRT380), SEQ ID NO: 13 (PRT410), SEQ ID NO: 14 (PRT525), or SEQ ID NO: 15 (PRT799), or (2-iv) an amino acid sequence having 90% or more sequence identity with the amino acid sequence set forth in SEQ ID NO: 12, SEQ ID NO: 13, SEQ ID NO: 14, or SEQ ID NO: 15.

Each of amino acid sequences set forth in SEQ ID NO: 16 (PRT313), SEQ ID NO: 12, SEQ ID NO: 13, SEQ ID NO: 14, and SEQ ID NO: 15 is obtained by adding the amino acid sequence set forth in SEQ ID NO: 11 (having a His tag sequence and a hinge sequence) to the N-terminus of each of the amino acid sequences set forth in SEQ ID NO: 10, SEQ ID NO: 6, SEQ ID NO: 7, SEQ ID NO: 8, and SEQ ID NO: 9.

The modified fibroin of (2-iii) may consist of the amino acid sequence set forth in SEQ ID NO: 12, SEQ ID NO: 13, SEQ ID NO: 14, or SEQ ID NO: 15.

The modified fibroin of (2-iv) may consist of an amino acid sequence having 90% or more sequence identity with the amino acid sequence set forth in SEQ ID NO: 12, SEQ ID NO: 13, SEQ ID NO: 14, or SEQ ID NO: 15. The modified fibroin of (2-iv) is also a protein containing the domain sequence represented by Formula 1: [(A)ₙ motif-REP]ₘ. The sequence identity is preferably 95% or more.

The modified fibroin of (2-iv) preferably has 90% or more sequence identity with the amino acid sequence set forth in SEQ ID NO: 12, SEQ ID NO: 13, SEQ ID NO: 14, or SEQ ID NO: 15, and z/w is preferably 50.9% or more, in which the total number of amino acid residues in the amino acid sequence consisting of XGX (where X represents the amino acid residue other than glycine) in the REP is z, and the total number of amino acid residues in the REP in the domain sequence is w.

The second modified fibroin may include a secretory signal for releasing the protein produced in the recombinant protein production system to the outside of a host. The sequence of the secretory signal can be appropriately set depending on the type of the host.

The domain sequence of the third modified fibroin has an amino acid sequence in which a content of an (A)ₙ motif is reduced, as compared with the naturally derived fibroin. It can be said that the domain sequence of the third modified fibroin has an amino acid sequence corresponding to an amino acid sequence in which at least one or a plurality of (A)ₙ motifs are deleted, as compared with the naturally derived fibroin.

The third modified fibroin may have an amino acid sequence corresponding to an amino acid sequence in which 10 to 40% of the (A)ₙ motifs are deleted from the naturally derived fibroin.

The third modified fibroin may have a domain sequence having an amino acid sequence corresponding to an amino acid sequence obtained by deleting one (A)ₙ motif of every one to three (A)ₙ motifs at least from the N-terminal side to the C-terminal side, as compared with the naturally derived fibroin.

The third modified fibroin may have a domain sequence having an amino acid sequence corresponding to an amino acid sequence obtained by repeating deletion of at least two consecutive (A)ₙ motifs and deletion of one (A)ₙ motif in this order from the N-terminal side to the C-terminal side, as compared with the naturally derived fibroin.

The third modified fibroin may have a domain sequence having an amino acid sequence corresponding to an amino acid sequence in which at least (A)ₙ motif every other two positions is deleted from the N-terminal side to the C-terminal side.

The third modified fibroin may contain a domain sequence represented by Formula 1: [(A)ₙ motif-REP]ₘ, and may have an amino acid sequence in which x/y may be 20% or more, 30% or more, 40% or more, or 50% or more, in which when the number of amino acid residues in REPs in two [(A)ₙ motif-REP] units adjacent to each other are sequentially compared from the N-terminal side to the C-terminal side, and then the number of amino acid residues in REP having a small number of amino acid residues is set as 1, a maximum value of the total value obtained by summing up the number of amino acid residues in the two adjacent [(A)ₙ motif-REP] units where the ratio of the number of amino acid residues in the other REP is 1.8 to 11.3 is x, and the total number of amino acid residues in the domain sequence is y The number of alanine residues with respect to the total number of amino acid residues in the (A)ₙ motif is 83% or more, preferably 86% or more, more preferably 90% or more, still more preferably 95% or more, and still more preferably 100% (which means that the (A)ₙ motif consists of only alanine residues).

A method of calculating x/y will be described in more detail with reference to FIG. 5. FIG. 5 illustrates a domain sequence excluding the N-terminal sequence and the C-terminal sequence from the modified fibroin. The domain sequence has a sequence of (A)ₙ motif-first REP (50 amino acid residues)−(A)ₙ motif-second REP (100 amino acid residues)−(A)ₙ motif-third REP (10 amino acid residues)−(A)ₙ motif-fourth REP (20 amino acid residues)−(A)ₙ motif-fifth REP (30 amino acid residues)−(A)ₙ motif from the N-terminal side (left side).

The two adjacent [(A)ₙ motif-REP] units are sequentially selected from the N-terminal side to the C-terminal side so as not to overlap. In this case, an unselected [(A)ₙ motif-REP] unit may exist. FIG. 5 illustrates a pattern 1 (a comparison between first REP and second REP and a comparison between third REP and fourth REP), a pattern 2 (a comparison between first REP and second REP and a comparison between fourth REP and fifth REP), a pattern 3 (a comparison between second REP and third REP and a comparison between fourth REP and fifth REP), and a pattern 4 (a comparison between first REP and second REP). There are other selection methods besides this.

Next, for each pattern, the number of amino acid residues in each REP in the selected two adjacent [(A)ₙ motif-REP] units is compared. The comparison is performed by determining the ratio of the number of amino acid residues of the other REP in a case where one REP having a smaller number of amino acid residues is defined as 1. For example, in a case of comparing the first REP (50 amino acid residues) and the second REP (100 amino acid residues), the ratio of the number of amino acid residues of the second REP is 100/50=2 in a case where the first REP having a smaller number of amino acid residues is defined as 1. Similarly, in a case of comparing the fourth REP (20 amino acid residues) and the fifth REP (30 amino acid residues), the ratio of the number of amino acid residues of the fifth REP is 30/20=1.5 in a case where the fourth REP having a smaller number of amino acid residues is defined as 1.

In FIG. 5, a set of [(A)ₙ motif-REP] units in which the ratio of the number of amino acid residues in the other REP when one REP having a smaller number of amino acid residues is 1 is 1.8 to 11.3 is indicated by a solid line. In the present specification, the ratio is referred to as a Giza ratio. A set of [(A)ₙ motif-REP] units in which the ratio of the number of amino acid residues in the other REP when one REP having a smaller number of amino acid residues is 1 is less than 1.8 or more than 11.3 is indicated by a dashed line.

In each pattern, the number of all amino acid residues in two adjacent [(A)ₙ motif-REP] units indicated by solid lines (including not only the number of amino acid residues in REP but also the number of amino acid residues in (A)ₙ motif) are summed up. Then, the total values thus summed up are compared and the total value in the patterns at which the total value is maximized (the maximum value of the total value) is x. In the example shown in FIG. 5, the total value of the pattern 1 is the maximum.

Then, x/y (%) can be calculated by dividing x by the total number of amino acid residues y of the domain sequence.

In the third modified fibroin, x/y is preferably 50% or more, more preferably 60% or more, still more preferably 65% or more, even still more preferably 70% or more, still further preferably 75% or more, and particularly preferably 80% or more. An upper limit of x/y is not particularly limited, but may be, for example, 100% or less. In a case where the Giza ratio is 1:1.9 to 11.3, x/y is preferably 89.6% or more; in a case where the Giza ratio is 1:1.8 to 3.4, x/y is preferably 77.1% or more; in a case where the Giza ratio is 1:1.9 to 8.4, x/y is preferably 75.9% or more; and in a case where the Giza ratio is 1:1.9 to 4.1, x/y is preferably 64.2% or more.

In a case where the third modified fibroin is a modified fibroin in which at least seven of a plurality of (A)ₙ motifs in the domain sequence consist of only alanine residues, x/y is preferably 46.4% or more, more preferably 50% or more, still more preferably 55% or more, even still more preferably 60% or more, still further preferably 70% or more, and particularly preferably 80% or more. The upper limit of x/y is not particularly limited, but may be 100% or less.

Here, x/y in the naturally derived fibroin will be described. First, as described above, 663 types of fibroins (415 types of fibroins derived from spiders among them) were extracted by confirming fibroins with amino acid sequence information registered in NCBI GenBank by an exemplified method. x/y was calculated by the above-described calculation method from the amino acid sequences of naturally derived fibroins consisting of a domain sequence represented by Formula 1: [(A)ₙ motif-REP]ₘ, among all the extracted fibroins. The results in a case where the Giza ratio is 1:1.9 to 4.1 are illustrated in FIG. 8.

In FIG. 7, the horizontal axis represents x/y (%), and the vertical axis represents a frequency. As is clear from FIG. 7, the values of x/y in the naturally derived fibroin are all smaller than 64.2% (the largest value is 64.14%).

The third modified fibroin can be obtained from, for example, a cloned gene sequence of naturally derived fibroin, by deleting one or a plurality of sequences encoding an (A)ₙ motif so that x/y is 64.2% or more. In addition, for example, the third modified fibroin can also be obtained, from the amino acid sequence of naturally derived fibroin, by designing an amino acid sequence corresponding to deletion of one or a plurality of (A)ₙ motifs so that x/y is 64.2% or more, and chemically synthesizing a nucleic acid encoding the designed amino acid sequence. In any case, in addition to the modification corresponding to deletion of the (A)ₙ motif from the amino acid sequence of the naturally derived fibroin, modification of the amino acid sequence corresponding to substitution, deletion, insertion, and/or addition of one or a plurality of amino acid residues may be performed.

A more specific example of the third modified fibroin can include a modified fibroin having (3-i) an amino acid sequence set forth in SEQ ID NO: 17 (Met-PRT399), SEQ ID NO: 7 (Met-PRT410), SEQ ID NO: 8 (Met-PRT525), or SEQ ID NO: 9 (Met-PRT799), or (3-ii) an amino acid sequence having 90% or more sequence identity with the amino acid sequence set forth in SEQ ID NO: 17, SEQ ID NO: 7, SEQ ID NO: 8, or SEQ ID NO: 9.

The modified fibroin of (3-i) will be described. The amino acid sequence set forth in SEQ ID NO: 17 is obtained by deleting every other two (A)ₙ motifs from the N-terminal side to the C-terminal side from the amino acid sequence set forth in SEQ ID NO: 10 (Met-PRT313) corresponding to the naturally derived fibroin and further inserting one [(A)ₙ motif-REP] before the C-terminal sequence. The amino acid sequence set forth in SEQ ID NO: 7, SEQ ID NO: 8, or SEQ ID NO: 9 is as described in the second modified fibroin.

The value of x/y in the amino acid sequence set forth in SEQ ID NO: 10 (corresponding to naturally derived fibroin) at a Giza ratio of 1:1.8 to 11.3 is 15.0%. Both the value of x/y in the amino acid sequence set forth in SEQ ID NO: 17 and the value of x/y in the amino acid sequence set forth in SEQ ID NO: 7 are 93.4%. The value of x/y in the amino acid sequence set forth in SEQ ID NO: 8 is 92.7%. The value of x/y in the amino acid sequence set forth in SEQ ID NO: 9 is 89.8%. The values of z/w in the amino acid sequences set forth in SEQ ID NO: 10, SEQ ID NO: 17, SEQ ID NO: 7, SEQ ID NO: 8, and SEQ ID NO: 9 are 46.8%, 56.2%, 70.1%, 66.1%, and 70.0%, respectively.

The modified fibroin of (3-i) may consist of the amino acid sequence set forth in SEQ ID NO: 17, SEQ ID NO: 7, SEQ ID NO: 8, or SEQ ID NO: 9.

The modified fibroin of (3-ii) may consist of an amino acid sequence having 90% or more sequence identity with the amino acid sequence set forth in SEQ ID NO: 17, SEQ ID NO: 7, SEQ ID NO: 8, or SEQ ID NO: 9. The modified fibroin of (3-ii) is also a protein containing the domain sequence represented by Formula 1: [(A)ₙ motif-REP]ₘ. The sequence identity is preferably 95% or more.

The modified fibroin of (3-ii) preferably has 90% or more sequence identity with the amino acid sequence set forth in SEQ ID NO: 17, SEQ ID NO: 7, SEQ ID NO: 8, or SEQ ID NO: 9, and x/y is preferably 64.2% or more, in which when the number of amino acid residues in REPs in two [(A)ₙ motif-REP] units adjacent to each other are sequentially compared from the N-terminal side to the C-terminal side, and then the number of amino acid residues in REP having a small number of amino acid residues is set as 1, a maximum value of the total value obtained by summing up the number of amino acid residues in the two adjacent [(A)ₙ motif-REP] units where the ratio of the number of amino acid residues in the other REP is 1.8 to 11.3 (the Giza ratio is 1:1.8 to 11.3) is x, and the total number of amino acid residues in the domain sequence is y.

The third modified fibroin may have the above-described tag sequence at either or both of the N-terminus and the C-terminus.

A more specific example of the modified fibroin having a tag sequence can include modified fibroin having (3-iii) an amino acid sequence set forth in SEQ ID NO: 18 (PRT399), SEQ ID NO: 13 (PRT410), SEQ ID NO: 14 (PRT525), or SEQ ID NO: 15 (PRT799), or (3-iv) an amino acid sequence having 90% or more sequence identity with the amino acid sequence set forth in SEQ ID NO: 18, SEQ ID NO: 13, SEQ ID NO: 14, or SEQ ID NO: 15.

Each of the amino acid sequences set forth in SEQ ID NO: 18, SEQ ID NO: 13, SEQ ID NO: 14, and SEQ ID NO: 15 is obtained by adding the amino acid sequence set forth in SEQ ID NO: 11 (having a His tag sequence and a hinge sequence) to the N-terminus of each of the amino acid sequences set forth in SEQ ID NO: 17, SEQ ID NO: 7, SEQ ID NO: 8, and SEQ ID NO: 9.

The modified fibroin of (3-iii) may consist of the amino acid sequence set forth in SEQ ID NO: 18, SEQ ID NO: 13, SEQ ID NO: 14, or SEQ ID NO: 15.

The modified fibroin of (3-iv) may consist of an amino acid sequence having 90% or more sequence identity with the amino acid sequence set forth in SEQ ID NO: 18, SEQ ID NO: 13, SEQ ID NO: 14, or SEQ ID NO: 15. The modified fibroin of (3-iv) is also a protein containing the domain sequence represented by Formula 1: [(A)ₙ motif-REP]ₘ. The sequence identity is preferably 95% or more.

The modified fibroin of (3-iv) preferably has 90% or more sequence identity with the amino acid sequence set forth in SEQ ID NO: 18, SEQ ID NO: 13, SEQ ID NO: 14, or SEQ ID NO: 15, and x/y is preferably 64.2% or more, in which when the number of amino acid residues in REPs in two [(A)ₙ motif-REP] units adjacent to each other are sequentially compared from the N-terminal side to the C-terminal side, and then the number of amino acid residues in REP having a small number of amino acid residues is set as 1, a maximum value of the total value obtained by summing up the number of amino acid residues in the two adjacent [(A)ₙ motif-REP] units where the ratio of the number of amino acid residues in the other REP is 1.8 to 11.3 is x, and the total number of amino acid residues in the domain sequence is y.

The third modified fibroin may include a secretory signal for releasing the protein produced in the recombinant protein production system to the outside of a host. The sequence of the secretory signal can be appropriately set depending on the type of the host.

The domain sequence of the fourth modified fibroin has an amino acid sequence in which a content of an (A)ₙ motif and a content of glycine residues are reduced, as compared with the naturally derived fibroin. It can be said that the domain sequence of the fourth modified fibroin has an amino acid sequence corresponding to an amino acid sequence in which at least one or a plurality of (A)ₙ motifs are deleted and at least one or a plurality of glycine residues in REP are substituted with another amino acid residue, as compared with the naturally derived fibroin. That is, the fourth modified fibroin is modified fibroin having the characteristics of the above-described second modified fibroin and third modified fibroin. Specific aspects and the like of the fourth modified fibroin are as in the descriptions for the second modified fibroin and the third modified fibroin.

A more specific example of the fourth modified fibroin can include modified fibroin having (4-i) an amino acid sequence set forth in SEQ ID NO: 7 (Met-PRT410), SEQ ID NO: 8 (Met-PRT525), SEQ ID NO: 9 (Met-PRT799), SEQ ID NO: 13 (PRT410), SEQ ID NO: 14 (PRT525), or SEQ ID NO: 15 (PRT799), or (4-ii) an amino acid sequence having 90% or more sequence identity with the amino acid sequence set forth in SEQ ID NO: 7, SEQ ID NO: 8, SEQ ID NO: 9, SEQ ID NO: 13, SEQ ID NO: 14, or SEQ ID NO: 15. Specific aspects of the modified fibroin having the amino acid sequence set forth in SEQ ID NO: 7, SEQ ID NO: 8, SEQ ID NO: 9, SEQ ID NO: 13, SEQ ID NO: 14, or SEQ ID NO: 15 are as described above.

The domain sequence of the fifth modified fibroin may have an amino acid sequence including a region having a locally high hydropathy index corresponding to an amino acid sequence in which one or a plurality of amino acid residues in REP are substituted with amino acid residues having a high hydropathy index and/or one or a plurality of amino acid residues having a high hydropathy index are inserted into REP, as compared with the naturally derived fibroin.

The region having a locally high hydropathy index preferably consists of consecutive two to four amino acid residues.

The above-described amino acid residue having a high hydropathy index is more preferably an amino acid residue selected from isoleucine (I), valine (V), leucine (L), phenylalanine (F), cysteine (C), methionine (M), and alanine (A).

The fifth modified fibroin may be further subjected to modification of an amino acid sequence corresponding to substitution, deletion, insertion, and/or addition of one or a plurality of amino acid residues as compared with the naturally derived fibroin, in addition to modification corresponding to substitution of one or a plurality of amino acid residues in REP with amino acid residues having a high hydropathy index and/or insertion of one or a plurality of amino acid residues having a high hydropathy index into REP, as compared with the naturally derived fibroin.

The fifth modified fibroin can be obtained by, for example, substituting one or a plurality of hydrophilic amino acid residues in REP (for example, amino acid residues having a negative hydropathy index) with hydrophobic amino acid residues (for example, amino acid residues having a positive hydropathy index) from a cloned gene sequence of naturally derived fibroin, and/or inserting one or a plurality of hydrophobic amino acid residues into REP. In addition, the fifth modified fibroin can be obtained by, for example, designing an amino acid sequence corresponding to substitution of one or a plurality of hydrophilic amino acid residues in REP with hydrophobic amino acid residues from an amino acid sequence of naturally derived fibroin, and/or insertion of one or a plurality of hydrophobic amino acid residues into REP, and chemically synthesizing a nucleic acid encoding the designed amino acid sequence. In any case, in addition to modification corresponding to substitution of one or a plurality of hydrophilic amino acid residues in REP with hydrophobic amino acid residues from amino acid sequences of naturally derived fibroin, and/or insertion of one or a plurality of hydrophobic amino acid residues into REP, modification of an amino acid sequence corresponding to substitution, deletion, insertion, and/or addition of one or a plurality of amino acid residues may be further performed.

The fifth modified fibroin may contain a domain sequence represented by Formula 1: [(A)ₙ motif-REP]ₘ, and may have an amino acid sequence in which p/q is 6.2% or more in a case where in all REPs included in a sequence excluding the sequence from the (A)ₙ motif located at the most C-terminal side to the C-terminus of the domain sequence from the domain sequence, the total number of amino acid residues included in a region where the average value of hydropathy indices of four consecutive amino acid residues is 2.6 or more is defined as p, and the total number of amino acid residues included in a sequence excluding the sequence from the (A)ₙ motif located at the most C-terminal side to the C-terminus of the domain sequence from the domain sequence is defined as q.

A known index (Hydropathy index: Kyte J, & Doolittle R (1982), “A simple method for displaying the hydropathic character of a protein”, J. Mol. Biol., 157, pp. 105-132) is used as the hydropathy index of the amino acid residue. Specifically, the hydropathy index (hereinafter, also referred to as “HI”) of each amino acid is as shown in Table 1.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Asparaginic acid (Asp) | −3.5 |
| Glutamine (Gln) | −3.5 |
| Glutamic acid (Glu) | −3.5 |
| Lysine (Lys) | −3.9 |
| Arginine (Arg) | −4.5 |

The calculation method of p/q will be described in more detail. In the calculation, a sequence excluding the sequence from the (A)ₙ motif located at the most C-terminal side to the C-terminus of the domain sequence from the domain sequence represented by Formula 1 [(A)ₙ motif-REP]ₘ (hereinafter also referred to as “sequence A”) is used. First, in all REPs contained in the sequence A, an average value of hydropathy indices of four consecutive amino acid residues is calculated. The average value of the hydropathy indices is determined by dividing the total sum of HIs of respective amino acid residues included in the four consecutive amino acid residues by 4 (number of amino acid residues). The average value of the hydropathy indices is determined for all of the four consecutive amino acid residues (each of the amino acid residues is used for calculating the average value 1 to 4 times). Next, a region where the average value of the hydropathy indices of the four consecutive amino acid residues is 2.6 or more is specified. Even in a case where certain amino acid residues correspond to a plurality of “four consecutive amino acid residues having an average value of hydropathy indices of 2.6 or more”, the amino acid residue is included as one amino acid residue in the region. The total number of amino acid residues included in the region is p. In addition, the total number of amino acid residues included in the sequence A is q.

For example, in a case where the “four consecutive amino acid residues having an average value of the hydropathy indices of 2.6 or more” are extracted from 20 places (no overlap), in the region where the average value of the hydropathy indices of four consecutive amino acid residues is 2.6 or more, 20 of the four consecutive amino acid residues (no overlap) are included, and thus p is 20×4=80. In addition, for example, in a case where two of the “four consecutive amino acid residues having an average value of the hydropathy indices of 2.6 or more” overlap by only one amino acid residue, in the region where the average value of the hydropathy indices of four consecutive amino acid residues is 2.6 or more, the number of amino acid residues is 7 (p=2×4−1=7, “−1” is the deduction of overlap). For example, in a case of the domain sequence shown in FIG. 8, there are seven “four consecutive amino acid residues having an average value of the hydropathy indices of 2.6 or more” without overlapping, and thus p is 7×4=28. In addition, for example, in a case of the domain sequence illustrated in FIG. 8, q is 4+50+4+40+4+10+4+20+4+30=170 (not including the (A)ₙ motif located at the end of the C-terminal side). Next, p/q (%) can be calculated by dividing p by q. In a case of FIG. 8, 28/170=16.47%.

In the fifth modified fibroin, p/q is preferably 6.2% or more, more preferably 7% or more, still more preferably 10% or more, even still more preferably 20% or more, and still further preferably 30% or more. An upper limit of p/q is not particularly limited, but may be 45% or less, for example.

The fifth modified fibroin can be obtained by, for example, substituting one or a plurality of hydrophilic amino acid residues in REP (for example, amino acid residues having a negative hydropathy index) with hydrophobic amino acid residues (for example, amino acid residues having a positive hydropathy index) so that a cloned amino acid sequence of naturally derived fibroin satisfies the condition of p/q, and/or modifying the cloned amino acid sequence of naturally derived fibroin with an amino acid sequence including a region having a locally high hydropathy index by inserting one or a plurality of hydrophobic amino acid residues into REP. In addition, the fifth modified fibroin can also be obtained by, for example, designing an amino acid sequence satisfying the condition of p/q from the amino acid sequence of the naturally derived fibroin, and chemically synthesizing a nucleic acid encoding the designed amino acid sequence. In any case, modification corresponding to substitution, deletion, insertion, and/or addition of one or a plurality of amino acid residues may also be performed, in addition to modification corresponding to substitution of one or a plurality of amino acid residues in REP with amino acid residues having a high hydropathy index, and/or insertion of one or a plurality of amino acid residues having a high hydropathy index into REP, as compared with the naturally derived fibroin.

The amino acid residue having a high hydropathy index is not particularly limited, but is preferably isoleucine (I), valine (V), leucine (L), phenylalanine (F), cysteine (C), methionine (M), and alanine (A), and more preferably valine (V), leucine (L), and isoleucine (I).

A more specific example of the fifth modified fibroin can include modified fibroin having (5-i) an amino acid sequence set forth in SEQ ID NO: 19 (Met-PRT720), SEQ ID NO: 20 (Met-PRT665), or SEQ ID NO: 21 (Met-PRT666), or (5-ii) an amino acid sequence having 90% or more sequence identity with the amino acid sequence set forth in SEQ ID NO: 19, SEQ ID NO: 20, or SEQ ID NO: 21.

The modified fibroin of (5-i) will be described. The amino acid sequence set forth in SEQ ID NO: 19 is obtained by inserting an amino acid sequence consisting of three amino acid residues (VLI) at two sites for each REP into the amino acid sequence set forth in SEQ ID NO: 7 (Met-PRT410), except for the domain sequence at the end on the C-terminal side, and further substituting a part of glutamine (Q) residues with serine (S) residues and deleting a part of amino acids on the C-terminal side. The amino acid sequence set forth in SEQ ID NO: 20 is obtained by inserting the amino acid sequence consisting of three amino acid residues (VLI) at one site for each REP into the amino acid sequence set forth in SEQ ID NO: 8 (Met-PRT525). The amino acid sequence set forth in SEQ ID NO: 21 is obtained by inserting the amino acid sequence consisting of three amino acid residues (VLI) at two sites for each REP into the amino acid sequence set forth in SEQ ID NO: 8.

The modified fibroin of (5-i) may consist of the amino acid sequence set forth in SEQ ID NO: 19, SEQ ID NO: 20, or SEQ ID NO: 21.

The modified fibroin of (5-ii) may consist of an amino acid sequence having 90% or more sequence identity with the amino acid sequence represented by SEQ ID NO: 19, SEQ ID NO: 20, or SEQ ID NO: 21.

The modified fibroin of (5-ii) is also a protein including a domain sequence represented by Formula 1: [(A)ₙ motif-REP]ₘ. The sequence identity is preferably 95% or more.

It is preferable that the modified fibroin of (5-ii) has a sequence identity of 90% or more with the amino acid sequence set forth in SEQ ID NO: 19, SEQ ID NO: 20, or SEQ ID NO: 21, and p/q is 6.2% or more in a case where in all REPs included in a sequence excluding the sequence from the (A)ₙ motif located at the most C-terminal side to the C-terminus of the domain sequence from the domain sequence, the total number of amino acid residues included in a region where the average value of hydropathy indices of four consecutive amino acid residues is 2.6 or more is defined as p, and the total number of amino acid residues included in a sequence excluding the sequence from the (A)ₙ motif located at the most the C-terminal side to the C-terminus of the domain sequence from the domain sequence is defined as q.

The fifth modified fibroin may have a tag sequence at either or both of the N-terminus and the C-terminus.

A more specific example of the modified fibroin having a tag sequence can include modified fibroin having (5-iii) an amino acid sequence set forth in SEQ ID NO: 22 (PRT720), SEQ ID NO: 23 (PRT665), or SEQ ID NO: 24 (PRT666), or (5-iv) an amino acid sequence having 90% or more sequence identity with the amino acid sequence set forth in SEQ ID NO: 22, SEQ ID NO: 23, or SEQ ID NO: 24.

Each of the amino acid sequences set forth in SEQ ID NO: 22, SEQ ID NO: 23, and SEQ ID NO: 24 is obtained by adding the amino acid sequence set forth in SEQ ID NO: 11 (having a His tag sequence and a hinge sequence) to the N-terminus of each of the amino acid sequences set forth in SEQ ID NO: 19, SEQ ID NO: 20, and SEQ ID NO: 21.

The modified fibroin of (5-iii) may consist of the amino acid sequence set forth in SEQ ID NO: 22, SEQ ID NO: 23, or SEQ ID NO: 24.

The modified fibroin of (5-iv) may consist of an amino acid sequence having 90% or more sequence identity with the amino acid sequence set forth in SEQ ID NO: 22, SEQ ID NO: 23, or SEQ ID NO: 24. The modified fibroin of (5-iv) is also a protein containing the domain sequence represented by Formula 1: [(A)ₙ motif-REP]ₘ. The sequence identity is preferably 95% or more.

The modified fibroin of (5-iv) preferably has 90% or more sequence identity with the amino acid sequence set forth in SEQ ID NO: 22, SEQ ID NO: 23, or SEQ ID NO: 24, and p/q is preferably 6.2% or more, in which in all REPs contained in a sequence excluding a sequence from a (A)ₙ motif located the most C-terminal side to the C-terminus of the domain sequence from the domain sequence, a total number of amino acid residues contained in a region where an average value of hydropathy indices of four consecutive amino acid residues is 2.6 or more is p, and a total number of amino acid residues contained in the sequence excluding the sequence from the (A)ₙ motif located the most C-terminal side to the C-terminus of the domain sequence from the domain sequence is q.

The fifth modified fibroin may include a secretory signal for releasing the protein produced in the recombinant protein production system to the outside of a host. The sequence of the secretory signal can be appropriately set depending on the type of the host.

The sixth modified fibroin has an amino acid sequence in which a content of glutamine residues is reduced, as compared with the naturally derived fibroin.

In the sixth modified fibroin, at least one motif selected from a GGX motif and a GPGXX motif is preferably included in the amino acid sequence of REP.

In a case where the sixth modified fibroin includes the GPGXX motif in REP, a GPGXX motif content rate is usually 1% or more, may also be 5% or more, and preferably 10% or more. An upper limit of the GPGXX motif content rate is not particularly limited, and may be 50% or less, or may also be 30% or less.

In the present specification, the “GPGXX motif content rate” is a value calculated by the following method. In fibroin (modified fibroin or naturally derived fibroin) containing a domain sequence represented by Formula 1: [(A)ₙ motif-REP]ₘ or Formula 2: [(A)ₙ motif-REP]ₘ−(A)ₙ motif, the GPGXX motif content rate is calculated as s/t, in which the number obtained by tripling the total number of GPGXX motifs in the regions of all REPs contained in a sequence excluding the sequence from the (A)ₙ motif located at the most C-terminal side to the C-terminus of the domain sequence from the domain sequence (that is, corresponding to the total number of G and P in the GPGXX motifs) is s, and the total number of amino acid residues in all REPs excluding the sequence from the (A)ₙ motif located at the most C-terminal side to the C-terminus of the domain sequence from the domain sequence and further excluding the (A)ₙ motifs is t.

For the calculation of the GPGXX motif content rate, the “sequence excluding a sequence from the (A)ₙ motif located at the most C-terminal side to the C-terminus of the domain sequence from the domain sequence” is used to exclude the effect occurring due to the fact that the “sequence from the (A)ₙ motif located at the most C-terminal side to the C-terminus of the domain sequence” (sequence corresponding to REP) may have a sequence having a low correlation with the sequence characteristic of fibroin, which influences the calculation result of the GPGXX motif content rate in a case where m is small (that is, in a case where the domain sequence is short). In a case where the “GPGXX motif” is located at the C-terminus of REP, it is regarded as the “GPGXX motif” even when “XX” is, for example, “AA”.

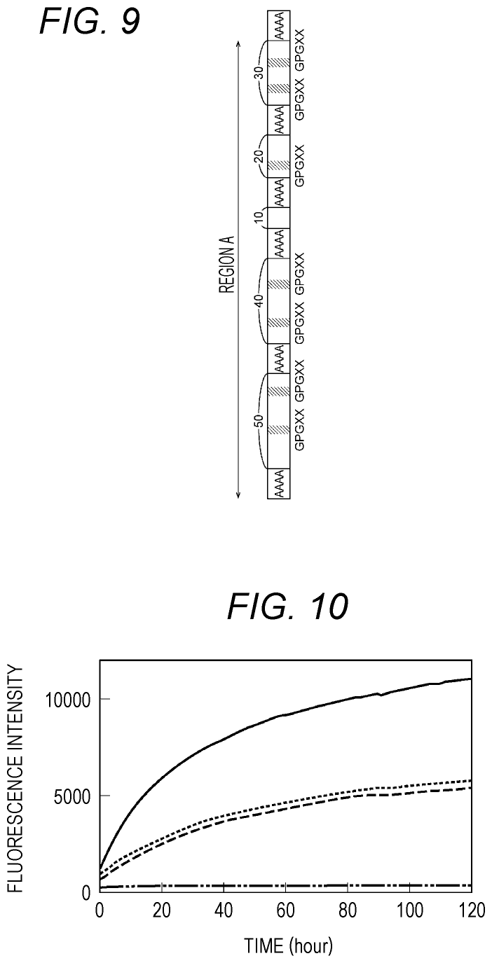

FIG. 9 is a schematic view illustrating an example of a domain sequence of modified fibroin. The calculation method of the GPGXX motif content rate will be specifically described with reference to FIG. 9. First, in the domain sequence of the modified fibroin (“[(A)ₙ motif-REP]ₘ−(A)ₙ motif” type) illustrated in FIG. 9, since all REPs are contained in the “sequence excluding the sequence from the (A)ₙ motif located at the most C-terminal side to the C-terminus of the domain sequence from the domain sequence” (the sequence indicated by the “region A” in FIG. 9), the number of GPGXX motifs for calculating s is 7, and s is 7×3=21. Similarly, all REPs are included in the sequence excluding the sequence from the (A)ₙ motif located at the most C-terminal side to the C-terminus of the domain sequence from the domain sequence” (the sequence indicated by the “region A” in FIG. 9). Thus, the total number t of amino acid residues in all REPs further excluding the (A)ₙ motifs from the sequence is 50+40+10+20+30=150. Next, s/t (%) can be calculated by dividing s by t, and in the case of the modified fibroin of FIG. 9, s/t (%) is 21/150=14.0%.

In the sixth modified fibroin, a glutamine residue content rate is preferably 9% or less, more preferably 7% or less, still more preferably 4% or less, and particularly preferably 0%.

In the present specification, the “glutamine residue content rate” is a value calculated by the following method. In fibroin (modified fibroin or naturally derived fibroin) containing a domain sequence represented by Formula 1: [(A)ₙ motif-REP]ₘ or Formula 2: [(A)ₙ motif-REP]ₘ−(A)ₙ motif, the glutamine residue content rate is calculated as u/t, in which a total number of glutamine residues in regions of all REPs contained in a sequence excluding the sequence from the (A)ₙ motif located at the most C-terminal side to the C-terminus of the domain sequence from the domain sequence (sequence corresponding to the “region A” in FIG. 9) is u, and a total number of amino acid residues in all REPs excluding the sequence from the (A)ₙ motif located at the most C-terminal side to the C-terminus of the domain sequence from the domain sequence and further excluding (A)ₙ motifs is t. In the calculation of the glutamine residue content rate, the reason for targeting the “sequence excluding the sequence from the (A)ₙ motif located at the most C-terminal side to the C-terminus of the domain sequence from the domain sequence” is the same as the reason described above.

The domain sequence of the sixth modified fibroin may have an amino acid sequence corresponding to deletion of one or a plurality of glutamine residues in REP, or substitution of one or a plurality of glutamine residues with another amino acid residue, as compared with the naturally derived fibroin.

“Another amino acid residue” may be an amino acid residue other than a glutamine residue, but is preferably an amino acid residue having a higher hydropathy index than that of a glutamine residue. The hydropathy index of the amino acid residue is as shown in Table 1.

As shown in Table 1, examples of the amino acid residue having a higher hydropathy index than that of the glutamine residue can include amino acid residues selected from isoleucine (I), valine (V), leucine (L), phenylalanine (F), cysteine (C), methionine (M), alanine (A), glycine (G), threonine (T), serine (S), tryptophan (W), tyrosine (Y), proline (P), and histidine (H). Among them, the amino acid residue is more preferably an amino acid residue selected from isoleucine (I), valine (V), leucine (L), phenylalanine (F), cysteine (C), methionine (M), and alanine (A), and still more preferably an amino acid residue selected from isoleucine (I), valine (V), leucine (L), and phenylalanine (F).

In the sixth modified fibroin, the hydrophobicity of REP is preferably −0.8 or more, more preferably −0.7 or more, still more preferably 0 or more, even still more preferably 0.3 or more, and particularly preferably 0.4 or more. An upper limit of the hydrophobicity of REP is not particularly limited, but may be 1.0 or less or 0.7 or less.

In the present specification, the “hydrophobicity of REP” is a value calculated by the following method. In fibroin (modified fibroin or naturally derived fibroin) containing a domain sequence represented by Formula 1: [(A)ₙ motif-REP]ₘ or Formula 2: [(A)ₙ motif-REP]ₘ−(A)ₙ motif, the hydrophobicity of REP is calculated as v/t, in which the sum of hydropathy indices of the amino acid residues in the regions of all REPs contained in the sequence excluding the sequence from the (A)ₙ motif located at the most C-terminal side to the C-terminus of the domain sequence from the domain sequence (sequence corresponding to the “region A” in FIG. 9) is v, and the total number of amino acid residues in all REPs excluding the sequence from the (A)ₙ motif located at the most C-terminal side to the C-terminus of the domain sequence from the domain sequence and further excluding (A)ₙ motifs is t. In the calculation of the hydrophobicity of REP, the reason for targeting the “sequence excluding the sequence from the (A)ₙ motif located at the most C-terminal side to the C-terminus of the domain sequence from the domain sequence” is the same as the reason described above.

The sixth modified fibroin may have a domain sequence that is further subjected to modification of an amino acid sequence corresponding to substitution, deletion, insertion, and/or addition of one or a plurality of amino acid residues, in addition to modification corresponding to deletion of one or a plurality of glutamine residues in REP, and/or substitution of one or a plurality of glutamine residues in REP with another amino acid residue, as compared to naturally derived fibroin.

The sixth modified fibroin can be obtained by, for example, deleting one or a plurality of glutamine residues in REP from a cloned gene sequence of naturally derived fibroin, and/or substituting one or a plurality of glutamine residues in REP with another amino acid residue. In addition, the sixth modified fibroin can be obtained by, for example, designing an amino acid sequence corresponding to deletion of one or a plurality of glutamine residues in REP from an amino acid sequence of naturally derived fibroin, and/or substitution of one or a plurality of glutamine residues in REP with another amino acid residue, and chemically synthesizing a nucleic acid encoding the designed amino acid sequence.

More specific examples of the sixth modified fibroin can include modified fibroin having (6-i) an amino acid sequence set forth in SEQ ID NO: 25 (Met-PRT888), SEQ ID NO: 26 (Met-PRT965), SEQ ID NO: 27 (Met-PRT889), SEQ ID NO: 28 (Met-PRT916), SEQ ID NO: 29 (Met-PRT918), SEQ ID NO: 30 (Met-PRT699), SEQ ID NO: 31 (Met-PRT698), SEQ ID NO: 32 (Met-PRT966), SEQ ID NO: 41 (Met-PRT917), or SEQ ID NO: 42 (Met-PRT1028), and modified fibroin having (6-ii) an amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin of (6-i) will be described. The amino acid sequence set forth in SEQ ID NO: 25 is obtained by substituting all QQs in the amino acid sequence set forth in SEQ ID NO: 7 (Met-PRT410) with VL. The amino acid sequence set forth in SEQ ID NO: 26 is obtained by substituting all QQs in the amino acid sequence set forth in SEQ ID NO: 7 with TS and substituting the remaining Q with A. The amino acid sequence set forth in SEQ ID NO: 27 is obtained by substituting all QQs in the amino acid sequence set forth in SEQ ID NO: 7 with VL and substituting the remaining Q with I. The amino acid sequence set forth in SEQ ID NO: 28 is obtained by substituting all QQs in the amino acid sequence set forth in SEQ ID NO: 7 with VI and substituting the remaining Q with L. The amino acid sequence set forth in SEQ ID NO: 29 is obtained by substituting all QQs in the amino acid sequence set forth in SEQ ID NO: 7 with VF and substituting the remaining Q with I.

The amino acid sequence set forth in SEQ ID NO: 30 is obtained by substituting all QQs in the amino acid sequence set forth in SEQ ID NO: 8 (Met-PRT525) with VL. The amino acid sequence set forth in SEQ ID NO: 31 is obtained by substituting all QQs in the amino acid sequence set forth in SEQ ID NO: 8 with VL and substituting the remaining Q with I.

The amino acid sequence set forth in SEQ ID NO: 32 is obtained by substituting, with VF, all QQs in a sequence obtained by repeating a region of 20 domain sequences present in the amino acid sequence set forth in SEQ ID NO: 7 (Met-PRT410) two times and substituting the remaining Q with I.

The amino acid sequence set forth in SEQ ID NO: 41 (Met-PRT917) is obtained by substituting all QQs in the amino acid sequence set forth in SEQ ID NO: 7 with LI and substituting the remaining Q with V. The amino acid sequence set forth in SEQ ID NO: 42 (Met-PRT1028) is obtained by substituting all QQs in the amino acid sequence set forth in SEQ ID NO: 7 with IF and substituting the remaining Q with T.

The glutamine residue content rate in each of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42 is 9% or less (Table 2).

TABLE 2

| | Glutamine | GPGXX | |
|---|---|---|---|
| | residue | motif | Hydrophobicity |
| Modified fibroin | content rate | content rate | of REP |
| Met-PRT410 (SEQ ID NO: 7) | 17.7% | 27.9% | −1.52 |
| Met-PRT888 (SEQ ID NO: 25) | 6.3% | 27.9% | −0.07 |
| Met-PRT965 (SEQ ID NO: 26) | 0.0% | 27.9% | −0.65 |
| Met-PRT889 (SEQ ID NO: 27) | 0.0% | 27.9% | 0.35 |
| Met-PRT916 (SEQ ID NO: 28) | 0.0% | 27.9% | 0.47 |
| Met-PRT918 (SEQ ID NO: 29) | 0.0% | 27.9% | 0.45 |
| Met-PRT699 (SEQ ID NO: 30) | 3.6% | 26.4% | −0.78 |
| Met-PRT698 (SEQ ID NO: 31) | 0.0% | 26.4% | −0.03 |
| Met-PRT966 (SEQ ID NO: 32) | 0.0% | 28.0% | 0.35 |
| Met-PRT917 (SEQ ID NO: 41) | 0.0% | 27.9% | 0.46 |
| Met-PRT1028 (SEQ ID NO: 42) | 0.0% | 28.1% | 0.05 |

The modified fibroin of (6-i) may consist of the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin of (6-ii) may consist of the amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42. The modified fibroin of (6-ii) is also a protein containing a domain sequence represented by Formula 1: [(A)ₙ motif-REP]ₘ or Formula 2: [(A)ₙ motif-REP]ₘ−(A)ₙ motif. The sequence identity is preferably 95% or more.

In the modified fibroin of (6-ii), a glutamine residue content rate is preferably 9% or less. In addition, in the modified fibroin of (6-ii), a GPGXX motif content rate is preferably 10% or more.

The sixth modified fibroin may have a tag sequence at either or both of the N-terminus and the C-terminus. This makes it possible to isolate, immobilize, detect, and visualize the modified fibroin.

More specific examples of the modified fibroin having a tag sequence can include modified fibroin having (6-iii) an amino acid sequence set forth in SEQ ID NO: 33 (PRT888), SEQ ID NO: 34 (PRT965), SEQ ID NO: 35 (PRT889), SEQ ID NO: 36 (PRT916), SEQ ID NO: 37 (PRT918), SEQ ID NO: 38 (PRT699), SEQ ID NO: 39 (PRT698), SEQ ID NO: 40 (PRT966), SEQ ID NO: 43 (PRT917), or SEQ ID NO: 44 (PRT1028), or modified fibroin having (6-iv) an amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44.

Each of the amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, and SEQ ID NO: 44 is obtained by adding the amino acid sequence set forth in SEQ ID NO: 11 (having a His tag sequence and a hinge sequence) to the N-terminus of each of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42. Since only the tag sequence is added to the N-terminus, the glutamine residue content rate is not changed, and the glutamine residue content rate in each of the amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44 is 9% or less (Table 3).

TABLE 3

| | Glutamine | GPGXX | |
|---|---|---|---|
| | residue | motif | Hydrophobicity |
| Modified fibroin | content rate | content rate | of REP |
| PRT888 (SEQ ID NO: 33) | 6.3% | 27.9% | −0.07 |
| PRT965 (SEQ ID NO: 34) | 0.0% | 27.9% | −0.65 |
| PRT889 (SEQ ID NO: 35) | 0.0% | 27.9% | 0.35 |
| PRT916 (SEQ ID NO: 36) | 0.0% | 27.9% | 0.47 |
| PRT918 (SEQ ID NO: 37) | 0.0% | 27.9% | 0.45 |
| PRT699 (SEQ ID NO: 38) | 3.6% | 26.4% | −0.78 |
| PRT698 (SEQ ID NO: 39) | 0.0% | 26.4% | −0.03 |
| PRT966 (SEQ ID NO: 40) | 0.0% | 28.0% | 0.35 |
| PRT917 (SEQ ID NO: 43) | 0.0% | 27.9% | 0.46 |
| PRT1028 (SEQ ID NO: 44) | 0.0% | 28.1% | 0.05 |

The modified fibroin of (6-iii) may consist of the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44.

The modified fibroin of (6-iv) may consist of the amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44. The modified fibroin of (6-iv) is also a protein containing a domain sequence represented by Formula 1: [(A)ₙ motif-REP]ₘ or Formula 2: [(A)ₙ motif-REP]ₘ−(A)ₙ motif. The sequence identity is preferably 95% or more.

In the modified fibroin of (6-iv), a glutamine residue content rate is preferably 9% or less. In addition, in the modified fibroin of (6-iv), a GPGXX motif content rate is preferably 10% or more.

The sixth modified fibroin may include a secretory signal for releasing the protein produced in the recombinant protein production system to the outside of a host. The sequence of the secretory signal can be appropriately set depending on the type of the host.

The modified fibroin may also be modified fibroin having at least two or more characteristics among the characteristics of the first modified fibroin, the second modified fibroin, the third modified fibroin, the fourth modified fibroin, the fifth modified fibroin, and the sixth modified fibroin.

The modified fibroin may be hydrophilic modified fibroin or hydrophobic modified fibroin. In the present specification, the “hydrophilic modified fibroin” is modified fibroin of which a value calculated by obtaining a sum of hydropathy indices (HIs) of all amino acid residues constituting the modified fibroin and then dividing the sum by a total number of amino acid residues (average HI) is 0 or smaller. The hydropathy index is as shown in Table 1. In addition, the “hydrophobic modified fibroin” is modified fibroin of which the average HI is larger than 0. Hydrophilic modified fibroin is particularly excellent in flame retardancy. Hydrophobic modified fibroin is particularly excellent in hygroscopic exothermicity and heat-retaining property.

Examples of the hydrophilic modified fibroin can include modified fibroin having an amino acid sequence set forth in SEQ ID NO: 4, an amino acid sequence set forth in SEQ ID NO: 6, SEQ ID NO: 7, SEQ ID NO: 8, or SEQ ID NO: 9, an amino acid sequence set forth in SEQ ID NO: 13, SEQ ID NO: 11, SEQ ID NO: 14, or SEQ ID NO: 15, an amino acid sequence set forth in SEQ ID NO: 18, SEQ ID NO: 7, SEQ ID NO: 8, or SEQ ID NO: 9, an amino acid sequence set forth in SEQ ID NO: 17, SEQ ID NO: 11, SEQ ID NO: 14, or SEQ ID NO: 15, or an amino acid sequence set forth in SEQ ID NO: 19, SEQ ID NO: 20, or SEQ ID NO: 21.

Examples of the hydrophobic modified fibroin can include modified fibroin having an amino acid sequence set forth in SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 43, or an amino acid sequence set forth in SEQ ID NO: 35, SEQ ID NO: 37, SEQ ID NO: 38, SEQ ID NO: 39, SEQ ID NO: 40, SEQ ID NO: 41, or SEQ ID NO: 44.

The protein according to the present embodiment can be produced by a normal method using a nucleic acid encoding the protein. The nucleic acid encoding the protein may be chemically synthesized based on base sequence information or may be synthesized using a PCR method or the like.

(Method for Producing Structural Protein Microbody)

A method for producing a structural protein microbody according to the present embodiment includes a first step of obtaining a structural protein solution containing a structural protein and a solubilizing agent (structural protein-containing solution), and a second step of reducing solubility of the protein in the structural protein solution to form a structural protein microbody. According to the production method, the structural protein microbody can be efficiently produced.

A specific method for obtaining a structural protein solution containing a structural protein and a solubilizing agent in the first step is not particularly limited. That is, examples of the method for obtaining a structural protein solution can include a method of adding (injecting) and dissolving a structural protein to and in a dissolving liquid obtained by dissolving a solubilizing agent in a predetermined solvent, a method of adding a structural protein to a predetermined solvent, adding a solubilizing agent thereto, and dissolving the solubilizing agent and the structural protein, and a method of simultaneously adding a structural protein and a solubilizing agent to a predetermined solvent and dissolving the solubilizing agent and the structural protein.

It is preferable that the structural protein is dissolved in the structural protein solution obtained in the first step to have a random coil structure. That is, the structural protein preferably forms a random coil structure in a solution. In addition, it is preferable that a solubilizing agent is dissolved in a structural protein solution together with a structural protein or a structural protein is dissolved in a solvent so that a random coil structure is formed. Furthermore, the solvent is preferably a solvent that can dissolve a structural protein so that a random coil structure is formed. Therefore, a protein microbody is more efficiently formed in the second step.

In addition, it is preferable that the structural protein is dissolved in the structural protein solution obtained in the first step so that the structural protein becomes a monomer (in a state where an aggregate is not formed). That is, it is preferable that the structural protein is dissolved in the solution as a monomer (in a state where an aggregate is not formed). In addition, it is preferable that the solubilizing agent can dissolve the structural protein in the solvent so that the structural protein becomes a monomer. Furthermore, the solvent is preferably a solvent that can dissolve a structural protein so that the structural protein becomes a monomer. Therefore, a protein microbody is more efficiently formed in the second step.

Here, the solvent of the structural protein solution is not particularly limited, and is preferably water from the viewpoint of easily adjusting the solubility of the protein. That is, the first step is preferably a step of dissolving a protein in an aqueous solution containing a solubilizing agent.

In addition, as such a solvent, a solvent in which a structural protein is sufficiently dissolved by a solubilizing agent so as to form a random coil structure is preferably used. This is due to the following reason.

That is, it has been found by the studies conducted by the present inventors that in a case where a solvent that can easily and sufficiently dissolve a structural protein, that is, a so-called good solvent is used without a special solubilizing agent, even when the structural protein is dissolved to form a random coil structure, efficient formation of a structural protein microbody in the second step may be difficult. Specifically, it is found that, for example, in a case where modified fibroin is used as a structural protein, when a protein solution is formed using a good solvent for modified fibroin, such as dimethyl sulfoxide, 1,1,1,3,3,3-hexafluoro-2-propanol, or formic acid, in the first step, a target structural protein microbody may not be easily formed in the second step. Therefore, in the first step, a poor solvent for the structural protein is preferably used as the solvent. Examples of such a solvent can include dimethyl sulfoxide, 1,1,1,3,3,3-hexafluoro-2-propanol, an organic solvent excluding formic acid, and water. These solvents are particularly preferably used in a case where modified fibroin or modified spider silk fibroin is used as the structural protein.

A raw material structural protein may be a structural protein exemplified as a structural protein constituting the above-described structural protein microbody. The form of the raw material structural protein is not particularly limited. The raw material structural protein is preferably in the form of powder, liquid, or the like, from the viewpoint of solubility.

The solubilizing agent may be any solubilizing agent that can solubilize a structural protein. As the solubilizing agent, a solubilizing agent that can solubilize a structural protein to form a random coil structure is preferable, and a solubilizing agent that can solubilize a structural protein so as to be dissolved as a monomer is preferable. As such a solubilizing agent, for example, dimethyl sulfoxide, 1,1,1,3,3,3-hexafluoro-2-propanol, guanidine hydrochloride (GuHCl), guanidine thiocyanate (GuSCN), sodium iodide, perchlorate, urea, or the like can be preferably used. In order to efficiently dissolve the structural protein to be formed as a monomer, it is desirable to dissolve the structural proteins that are aggregated and hardly dissolved, and as the solubilizing agent, dimethyl sulfoxide, 1,1,1,3,3,3-hexafluoro-2-propanol, guanidine hydrochloride (GuHCl), guanidine thiocyanate (GuSCN), sodium iodide, or perchlorate is particularly preferable.

The amount of the solubilizing agent is not particularly limited. A concentration of the solubilizing agent in the dissolving liquid may be, for example, 1 M to 8 M, and is preferably 3 M to 7 M and more preferably 4 M to 6 M.

A method for dissolving the structural protein is not particularly limited, and may be preferably selected from known methods. For example, the protein may be dissolved by shaking, stirring, an ultrasonic treatment, heating, or the like.

A concentration of the structural protein in the structural protein solution is, for example, 0.1 to 700 mg/mL, preferably 1 to 500 mg/mL, and more preferably 3 to 300 mg/mL.

Hereinafter, the first step will be specifically described with reference to a case where guanidine thiocyanate is used as the solubilizing agent. First, 1,000 μL of a 5 M aqueous guanidine thiocyanate solution is added to 100 mg of a structural protein powder, and the mixture is shaken (1,800 rpm) for 5 minutes. An ultrasonic treatment (for example, 20 to 30%, 10 seconds, 4 or 5 times, interval of 5 to 10 minutes) may be performed, if necessary. Whether or not the structural protein is dissolved can be confirmed by, for example, an ultraviolet-visible absorption measurement or the like.

In the first step, after the structural protein is dissolved, impurities may be removed by filter filtration or the like. The filter filtration method is not particularly limited, and an example thereof can include filtration using a filter (Ultrafree-MC-GV, Durapore, PVDF, 0.22 μm). In order to prevent clogging, the treatment by the filter filtration may be performed at, for example, 50 μL/sec or less.

<Second Step>

In the second step, the solubility of the structural protein in the structural protein solution is reduced to form a structural protein microbody. Here, a mechanism of forming the structural protein microbody is not necessarily clear, but it is presumed that the structural proteins (in particular, the structural proteins having a random coil structure) contained in the structural protein solution are aggregated by reducing the solubility in the structural protein solution, and a structural protein microbody is formed by the aggregate.

Examples of a method of reducing the solubility of the structural protein in the structural protein solution in the second step can include a method of adjusting a temperature of a structural protein solution to be lowered and increased, and a method of adding water, a surfactant, an organic solvent, an inorganic salt, or the like to a structural protein solution.

In addition, as another method, it is preferable to reduce the solubility of the structural protein by reducing the concentration of the solubilizing agent in the structural protein solution. The concentration of the solubilizing agent can be reduced by addition of water, addition of an organic solvent, or the like.

In the second step, it is preferable to reduce the solubility of the structural protein in the structural protein solution by combining two or more methods selected from the group consisting of temperature adjustment, addition of water, addition of a surfactant, addition of an organic solvent, and addition of an inorganic salt described above. As such, a plurality of methods are combined, such that the solubility can be finely adjusted and the above-described structural protein microbody can be easily obtained. More specifically, when the reduction in solubility is too small, the amount of structural protein microbody to be obtained is small, and when the reduction in solubility is too large, the amount of structural protein microbody to be obtained by aggregate is too small. Therefore, the method capable of more finely adjusting the solubility is preferable. In the second step, it is more preferable to combine two or more methods of addition of water, addition of a surfactant, addition of an organic solvent, and addition of an inorganic salt, and it is still more preferable to perform at least addition of water and addition of an organic solvent.

The organic solvent is preferably a solvent compatible with the solvent (for example, water) in the structural protein solution. Examples of the organic solvent can include alcohols such as methanol, ethanol, and 2-propanol; ketones such as acetone and 2-butanone; ethers such as tetrahydrofuran and 1,4-dioxane; and nitriles such as acetonitrile.

Examples of the inorganic salt can include ammonium sulfate, potassium acetate, and sodium chloride.

Examples of the surfactant can include octylphenol ethoxylate (for example, “Triton X-100” or the like, manufactured by Sigma-Aldrich Co., LLC.) and sodium dodecyl sulfate (SDS).

The water, the surfactant, the organic solvent, and the inorganic salt described above can also be collectively referred to as a dissolution inhibitor. An addition amount of the dissolution inhibitor may be appropriately adjusted depending on, for example, the concentration of the structural protein in the structural protein solution, the concentration of the solubilizing agent, the type of the solubilizing agent, the type of the dissolution inhibitor, the type of the dissolution inhibitor, and the like so that a generation amount of a target structural protein microbody is further increased.

It is preferable that the solution is homogenized by stirring, shaking, or the like after the addition of the dissolution inhibitor. For example, after the addition of the dissolution inhibitor, the solution can be homogenized by shaking the solution at 1,800 rpm for 5 minutes. In addition, the homogenized solution is allowed to stand for a predetermined time to more easily form a structural protein microbody. The standing time is not particularly, and may be, for example, about one day.

By reducing the solubility of the structural protein in the structural protein solution, a structural protein microbody is formed and a dispersion containing the structural protein microbody is obtained.

An example of the method of reducing the solubility of the structural protein in the structural protein solution in the second step can also include a method of applying a physical force such as a shear stress or a compressive stress to the structural protein solution, in addition to the above-described methods. In such a method of applying a shear stress or a compressive stress, for example, unlike the case of using the inhibitor described above, it is not necessary to remove the dissolution inhibitor in the subsequent step, and a structural protein microbody can be obtained more easily.

The method of applying a shear stress to the structural protein solution is not particularly limited, and for example, the structural protein solution may be swirled at a high speed and vigorously stirred using a vortex mixer or the like, the solution may be rotated and vigorously stirred using a stirring blade, or the solution may be passed through a narrow space such as a capillary at a high speed. In the case of applying a shear stress by rotating the structural protein solution at a high speed, the rotation time can be shortened as the rotation speed is higher, but for example, the structural protein solution is preferably rotated at 500 rpm or more for 78 hours or longer, more preferably rotated at 1,800 rpm or more for 2 hours or longer, and still more preferably rotated at 3,400 rpm or more for 30 minutes or longer.

The presence or absence of formation of the structural protein microbody can be observed, for example, by a fluorescence intensity measurement by ThT staining. Specifically, for example, a sample obtained by adding ThT to a structural protein solution is used as a measurement sample, and a fluorescence intensity is measured by a fluorometer, such that a formation state of a structural protein microbody in the structural protein solution can be observed.

An addition amount of ThT may be, for example, 4 μM.

The conditions of the fluorescence intensity measurement may be, for example, the conditions described in <(i) Fluorescence Intensity Measurement by thioflavin T staining (ThT staining)>.

The plate reader can follow a temporal change in fluorescence intensity. As the plate reader, for example, SYNERGY HTX (manufactured by BIOTEC Co., Ltd.) or the like can be used. The measurement may be performed according to the manual attached to the device.

An increase in fluorescence intensity of thioflavin T based on formation of a β-sheet structure is confirmed by a fluorometer, such that the formation of the structural protein microbody is confirmed. In addition, the formation of the β-sheet structure over time can also be followed with the plate reader. Furthermore, an optimal dilution condition can be determined by this analysis.

The formed structural protein microbody is dispersed and precipitated in the dispersion, and can be collected by a known method such as centrifugation or filter filtration. That is, the production method according to the present embodiment may further include a colleting step of colleting the structural protein microbody from the dispersion containing the structural protein microbody.

The colleting step may be a step of separating the dispersion into the structural protein microbody and the supernatant. The supernatant may contain a protein that did not form a structural protein microbody as a random coil.

The colleting step can be performed by a known method such as centrifugation or filter filtration. The conditions in the colleting step are not particularly limited. As an example of a case in which the colleting step is performed by centrifugation, centrifugation can be performed for 30 minutes under conditions of 20° C. and 14,500 rpm using a centrifuge (KUBOTA 3740, manufactured by KUBOTA Manufacturing Corporation) to separate the dispersion into the structural protein microbody and the supernatant, and the structural protein microbody can be collected.

The collected structural protein microbody may be dried, dispersed in a dispersion medium, and stored. As the dispersion medium, for example, an aqueous urea solution or the like can be preferably used.

(Method for Producing Nanofiber)

The structural protein microbody according to the present embodiment functions as a core for forming a protein nanofiber. Therefore, for example, the structural protein microbody is brought into contact with a solution in which a protein is dissolved, such that the protein is self-organized using the structural protein microbody as a core to form a protein nanofiber. That is, the method for producing a nanofiber according to the present embodiment includes step A of preparing a protein solution in which a protein is dissolved; and step B of mixing the protein solution with the structural protein microbody to obtain a protein nanofiber.

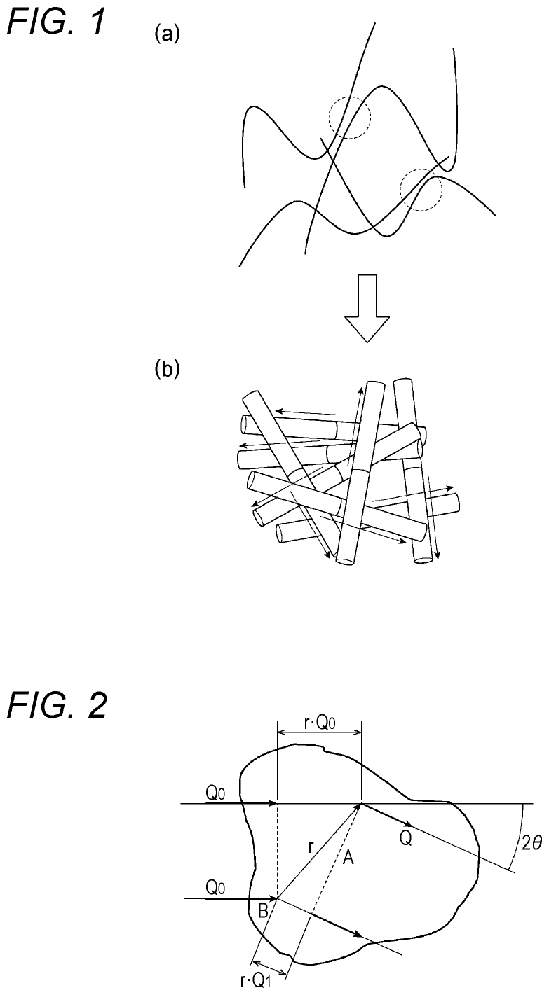

As illustrated in FIG. 1, the structural proteins do not form a steric structure in a completely dissolved state, and the structural proteins are only partially in contact with each other (portions indicated by circles with dashed lines in FIG. 1(a)). It is considered that cylindrical nanofibers as shown in FIG. 1(b) are formed by self-organization of the protein in the presence of the structural protein microbody.

In the present specification, the nanofiber refers to a fibrous substance having a diameter of 1 nm to 100 nm and a length larger than the diameter (for example, the length is 10 times or more greater than the diameter). The nanofiber may also be referred to as a fibril, a nanorod, or the like.

Step A may be a step of dissolving a protein in a first solvent to obtain a protein solution. In addition, step A may be a step of preparing an existing protein solution. An example of the protein used here can include a structural protein constituting the above-described structural protein microbody. In addition, an example of the protein can include a protein that can be used for industrial use or medical use, in addition to such a structural protein. Specific examples of such a protein can include an enzyme, a regulatory protein, a receptor, a peptide hormone, a cytokine, a membrane or transport protein, an antigen used for vaccination, a vaccine, an antigen-binding protein, an immunostimulatory protein, an allergen, and a full length antibody or an antibody fragment or a derivative thereof. The first solvent may be a solvent capable of dissolving a protein, and may be, for example, an organic solvent, a salt solution, an acidic solution, a basic solution, a chaotropic solution, or the like.

Examples of the organic solvent can include 1,1,1,3,3,3-hexafluoro-2-propanol, dimethyl sulfoxide, dimethylformamide, and N-methylpyrrolidone.

The salt solution may be, for example, an aqueous solution containing a salt. Examples of the salt can include sodium chloride, zinc chloride, and lithium chloride.

The acidic solution may be, for example, an aqueous solution containing an acid. Examples of the acid can include hydrochloric acid and acetic acid.

The basic solution may be, for example, an aqueous solution containing a base. Examples of the base can include sodium hydroxide, potassium hydroxide, and ammonia.

The chaotropic solution may be, for example, an aqueous solution containing a chaotropic agent. Examples of the chaotropic agent can include urea, guanidine hydrochloride, and guanidine thiocyanate.

A concentration of the protein in the protein solution is not particularly limited. The concentration of the protein in the protein solution may be, for example, 0.01 mass % or more, preferably 0.1 mass % or more, and still more preferably 1 mass % or more. In addition, the concentration of the protein in the protein solution may be, for example, 50 mass % or less, preferably 30 mass % or less, and still more preferably 25 mass % or less.

In step B, the protein solution and the protein microbody are mixed with each other. Therefore, the protein is self-organized using the protein microbody as a core, and a nanofiber is thus formed.

In step B, a mixing method is not particularly limited. Step B may be, for example, a step of mixing a protein solution with a powdery protein microbody, or a step of mixing a protein solution with a dispersion containing a protein microbody.

In step B, a mass ratio (C₁/C₀) of a content C₁ of the protein microbody to a content C₀ of the protein in the protein solution may be, for example, 0.01 or more, and is preferably 0.05 or more and more preferably 0.1 or more. In addition, the mass ratio (C₁/C₀) may be, for example, 100 or less, and is preferably 10 or less and more preferably 1 or less.

In step B, a mixed solution obtained by mixing a protein solution with a protein microbody may be allowed to stand for a predetermined time. Therefore, a yield of the nanofiber is further improved. The standing time is not particularly limited, and may be, for example, 3 minutes or longer, and is preferably 10 minutes or longer.

In step B, the mixed solution obtained by mixing the protein solution with the protein microbody is allowed to stand, if necessary, and then, a dissolution inhibitor may be added to the mixed solution. Therefore, the nanofibers are easily precipitated, and the nanofibers are more easily collected. Examples of the dissolution inhibitor can include ethanol and ammonium sulfate.

In step B, a method for collecting the nanofibers formed in the mixed solution is not particularly limited. For example, the nanofibers can be collected by a method such as centrifugation or filter filtration.

(Method for Producing Protein Structure)

A method for producing a protein structure according to the present embodiment includes step (a) of preparing a structural precursor containing a fibrous substance containing a protein; and step (b) of applying an anisotropic stress to the structural precursor to obtain a protein structure. According to such a production method, a protein structure containing a plurality of protein nanofibers oriented in one direction can be easily produced.

The fibrous substance may be a fibrous substance (a) containing the structural protein microbody described above, a fibrous substance (b) containing a self-organized protein using the structural protein microbody as a core, or both fibrous substances (a) and (b). In other words, the fibrous substance may be a protein nanofiber (corresponding to (b)), a precursor of a protein nanofiber (corresponding to (a)), or both of them. When the fibrous substance is a precursor of a protein nanofiber, the proteins are further aggregated and self-organized into a fibrous substance to form a protein nanofiber.

Examples of the proteins self-organized using the structural protein microbody or the proteins further aggregated and self-organized into a fibrous substance that constitute the fibrous substance (b) can include the structural protein constituting the structural protein microbody and the protein that can be used for industrial use or medical use.

The structural protein microbody may function as a core for forming a fibrous substance. For example, the structural protein microbody is brought into contact with a solution in which a protein is dissolved, such that the protein is self-organized using the structural protein microbody as a core to form a fibrous substance. In addition, the structural protein microbody is generated in the structural protein solution, such that a fibrous substance obtained using the structural protein microbody as a core can be formed.

(Structural Precursor)

In step (a), a structural precursor containing a fibrous substance is prepared. The structural precursor is not particularly limited as long as it is in a form capable of applying an anisotropic stress, and may be, for example, a hydrogel, a fiber, an aggregate, or a film.

The structural precursor is preferably shrunk over time from the viewpoint of easily applying an anisotropic stress.

Hereinafter, a method for obtaining a hydrogel containing a fibrous substance as a structural precursor will be described.

The hydrogel can be produced, for example, by diluting a protein-containing solution (preferably, the protein-containing solution in step (a) of the method for producing a protein microbody) by dialysis.

A low concentration solution having a lower concentration of a solubilizing agent than that of the protein-containing solution, a diluent containing no solubilizing agent, or the like can be used for the dilution in dialysis. Each of the low concentration solution and the diluent may be a buffer (buffer solution).

In a process of diluting the protein-containing solution stepwise by dialysis, a hydrogel is formed. In this case, a fibrous substance is formed in the protein-containing solution by dilution using a protein microbody as a core.

The hydrogel contains a fibrous substance. In addition, the hydrogel may further contain a random coil-shaped protein that is not self-organized.

(Step (b))

In step (b), an anisotropic stress is applied to the structural precursor. Therefore, a protein structure in which a plurality of protein nanofibers are oriented in one direction is formed.

A method of applying an anisotropic stress is not particularly limited, and examples thereof can include a method of using shrinkage over time, a method using a tensile tester, and a method using a stretching machine.

In the case of using shrinkage over time, for example, an anisotropic stress can be applied to the structural precursor by fixing both ends of the structural precursor in one direction and shrinking the structural precursor while maintaining the fixing.

For example, in the case where the structural precursor is a hydrogel, the hydrogel is dried in a state where both ends thereof are fixed in one direction, such that the hydrogel can be shrunk and an anisotropic stress can be applied to the hydrogel.

In the protein structure formed in step (b), an orientation state of the protein nanofiber in the structure can be confirmed by wide angle X-ray diffraction XRD. By the orientation of the protein nanofiber, a sharp peak is observed in a one-dimensional X-ray diffraction profile, and a sharp diffraction line is observed in a two-dimensional X-ray diffraction profile. In addition, a peak is observed at a specific azimuthal angle from an azimuthal angle distribution of the intensity obtained by circularly multiplying the specific diffraction angle. A crystal structure or the like of the protein in the protein structure can be confirmed by the peak.

The wide angle X-ray diffraction XRD measurement can be performed, for example, under the following conditions. Measuring apparatus: X-ray generator MicroMAX007 (manufactured by Rigaku Corporation), R-AXIS-IV (manufactured by Rigaku Corporation), measurement conditions: X-ray wavelength of 1.5418 Å (CuKα), room temperature (20° C.), camera length: 80 mm, exposure time of 15 minutes

In addition, the orientation state of the protein nanofiber in the protein structure can be observed using an atomic force microscope (AFM). In other words, according to the production method according to the present embodiment, it is possible to obtain a protein structure in which the protein nanofiber is highly oriented at a level at which the orientation state can be observed with AFM.

In the present embodiment, the protein nanofiber in the protein structure may have an amyloid-like crystal. The fact that the protein nanofiber has an amyloid-like crystal can be confirmed by an XRD measurement of the protein structure. Specifically, in a case where the protein nanofiber in the protein structure has an amyloid-like crystal, in a diffraction intensity profile obtained by XRD measurement, peaks close to the amyloid fiber (for example, peaks at 2θ=8° to 10° and 18° to 19.5°) are observed.

In addition, in the present embodiment, the protein nanofiber in the protein structure may have a poly-Ala-like crystal. The fact that the protein nanofiber has a poly-Ala-like crystal can be confirmed by an XRD measurement of the protein structure. Specifically, in a case where the protein nanofiber in the protein structure has a poly-Ala-like crystal, characteristic peaks of the poly-Ala-like crystal (for example, peaks at 2θ=15° to 17°, 18.5° to 20.5°, and 22.5° to 25.5°) in a diffraction intensity profile obtained by XRD measurement, and peaks (for example, peaks at β=75° to 105° and 255° to 285°, β=75° to 105° and 255° to 285°, and β=30° to 60°, 120° to 150°, 210° to 240°, and 300° to 330°) in an azimuthal angle intensity profile are observed, respectively.

In a case where the protein nanofiber has an amyloid-like crystal, β-strands in the amyloid-like crystal are preferably oriented perpendicular to the orientation direction of the protein nanofiber. In addition, in a case where the protein nanofiber has a poly-Ala-like crystal, β-strands in the poly-Ala-like crystal are preferably oriented parallel to the orientation direction of the protein nanofiber. Such a structure can be confirmed, for example, by an azimuthal angle distribution of intensity obtained by circularly multiplying a specific diffraction angle in a two-dimensional diffraction image of XRD measurement.

A thickness (diameter) of the protein nanofiber in the protein structure may be, for example, 1 nm or more, and is preferably 3 nm or more. In addition, a thickness (diameter) of the protein nanofiber may be, for example, 1,000 nm or less, and is preferably 500 nm or less.

A length of the protein nanofiber in the protein structure may be, for example, 10 nm or more, and is preferably 30 nm or more.

In the protein structure, the protein nanofibers may be linked to each other to form a long fiber, and may be bound to each other to form a fiber bundle.

The protein structure produced by the production method according to the present embodiment can be applied to various fields such as a cell sheet, a biomolecular device, a filter, spinning, and a cosmetic.

Although the preferred embodiment of the present invention has been described above, the present invention is not limited to the above embodiment.

### EXAMPLES

Hereinafter, although the present invention will be described in more detail by Examples, the present invention is not limited to these Examples.

### Example 1

A powder sample of fibroin having an amino acid sequence set forth in SEQ ID NO: 13 was prepared. To 300 mg of the powder sample, 3 mL of a guanidine thiocyanate buffer (5 M guanidine thiocyanate, 10 mM TrisHCl, pH 7.0) was added, and the mixture was shaken (1,800 rpm) for 5 minutes, thereby obtaining a structural protein solution (fibroin solution). Ultraviolet-visible absorption of the obtained fibroin solution was measured by NanoDrop (registered trademark). As a result of the measurement, in an ultraviolet-visible absorption spectrum, an absorption spectrum having a maximum at 280 nm was shown and no remarkable scattering was observed. It was confirmed from the result that the fibroin was not completely dissolved.

Next, 9 mL of ethanol was added to the structural protein solution while the structural protein solution was stirred (1,800 rpm, 5 minutes) with a vortex mixer so that the final concentration was 75 vol % (that is, in order to obtain 4-fold dilution). Therefore, a structural protein microbody was formed in the solution. Centrifugation was performed under conditions of 15,000 g, 10 minutes, and 20° C. using a centrifuge (KUBOTA 3740, manufactured by KUBOTA Manufacturing Corporation) to collect the structural protein microbody as a precipitated fraction. Thereafter, washing with ultrapure water and lyophilization were performed to obtain 253.8 mg of a structural protein microbody.

The obtained structural protein microbody was subjected to a fluorescence intensity measurement by ThT staining, small angle X-ray scattering (SAXS) analysis, Guinier analysis, and a measurement of an average particle size by a dynamic light scattering method under the following methods.

<Fluorescence Intensity Measurement by ThT Staining>

A measurement sample obtained by dispersing the structural protein microbody in a dispersion (aqueous solution of 6 M urea, 10 mM TrisHCl, and 5 mM DTT, pH 7.0) at a concentration of 5 mg/mL and further adding 4 μM of ThT was used. The measurement conditions were as follows.

Measuring instrument: JASCO FP-8200 (manufactured by JASCO Corporation), measurement range: 440 to 600 nm, excitation wavelength: 450 nm, scan speed: medium, number of times of measurement: three times

The result of the fluorescence intensity measurement by ThT staining is indicated by A1 (solid line) in FIG. 3. A1 in FIG. 3 has a peak within a range of 480 to 500 nm. It was confirmed from this that the structural protein microbody obtained in Example 1 had a β-sheet structure.

<SAXS Measurement>

A measurement sample obtained by dispersing the structural protein microbody in a dispersion (aqueous solution of 6 M urea, 10 mM TrisHCl, and 5 mM DTT, pH 7.0) at a concentration of 5 mg/mL and further adding 4 μM of ThT was used. The measurement conditions were as follows. Measuring apparatus: X-ray small angle scattering measuring apparatus NANO-Viewer (manufactured by Rigaku Corporation), X-ray generator MicroMAX007 (manufactured by Rigaku Corporation), detector PILATUS 200K (manufactured by DECTRIS Ltd.), measurement conditions: X-ray wavelength of 1.5418 Å (CuKα), room temperature (20° C.), exposure time of 30 minutes

After the measurement was performed under the above conditions, circumferential averaging was performed to obtain a one-dimensional profile. A modified Kratky plot was obtained by analyzing the one-dimensional profile using IgorPro software (manufactured by WaveMetrics Inc.). The obtained modified Kratky plot is indicated by A2 (solid line) in FIG. 4. A2 of FIG. 4 has a peak in a region where Q is 0.15 or less. In addition, a change width in a region where Q is 0.15 or more and 0.3 or less is ±10% or less. It was confirmed from this result that the structural protein microbody had a core portion having a high electron density and a random coil disposed to surround the core portion.

<Guinier Analysis>

Guinier analysis was performed as described in (iii) Aggregate of structural protein molecules. As a result, it was confirmed that the origin scattering intensity obtained from the first measurement sample group was 20.617, the origin scattering intensity obtained from the second measurement sample group was 7.38, and the structural protein microbody was an aggregate of three structural protein molecules.

<Measurement of Average Particle Size by Dynamic Light Scattering Method>

As a measurement sample group, measurement samples were prepared by dispersing structural protein microbodies in first dispersions (aqueous solution of 6 M urea, 10 mM TrisHCl, and 5 mM DTT, pH 7.0) at concentrations of 2 mg/mL, 4 mg/mL, 6 mg/mL, 8 mg/mL, and 10 mg/mL, respectively. Next, a particle size distribution of each of the measurement samples was measured by a dynamic light scattering method under the following conditions to determine a volume average size. Measuring apparatus: ZETASIZER nano-ZS (manufactured by Malvern Panalytical), measurement temperature: 20° C.

The measurement was performed 5 times for each measurement sample to determine an average value of the obtained measured values. From the concentration and the measured value (average value) of each of the measurement samples, a plot of the average particle size against the concentration was obtained, and 0 concentration extrapolation excluding an intermolecular interaction was performed. The value obtained by the 0 concentration extrapolation was defined as an average particle size of the structural protein microbodies.

As a result of the measurement, the average particle size of the structural protein microbodies was 13.225 nm.

Next, a nanofiber was produced using the obtained structural protein microbody.

Specifically, first, 8.33 mg of a protein powder (powder of fibroin having an amino acid sequence set forth in SEQ ID NO: 13) was added to 1 ml of an aqueous guanidine thiocyanate solution (5 M guanidine thiocyanate, 10 mM TrisHCl, 5 mM DTT, pH 7.0), and the mixture was stirred with a vortex mixer for 1 minute. The operation was performed 6 times, and the amount of powder added was 50 mg. Thereafter, the mixture was allowed to stand at room temperature for one day. After the standing, centrifugation (20,000 g, 20 minutes, 20° C.) was performed using a centrifuge (KUBOTA 3740), and the supernatant was collected. Thereafter, the sample solution was placed in a dialysis tube (#D100, manufactured by BioDesign Inc.) and dialyzed with a 6 M urea solution for two days (external liquid exchange was performed three times). Therefore, a protein solution (S₀) (concentration of protein: 7.5 mg/mL) containing no structural protein microbody was obtained. Next, the structural protein microbody was dispersed in a dispersion (aqueous solution of 6 M urea, 10 mM TrisHCl, and 5 mM DTT, pH of 7.0) at a concentration of 7.5 mg/mL to obtain a protein solution (S₁) containing a structural protein microbody.

The solution (S₀) and the solution (S₁) were mixed in S₀:S₁=1:2 (volume ratio) and the mixture was diluted 2-fold with a diluent (10 mM TrisHCl, 5 mM DTT, pH 7.0) to obtain a nanofiber.

Volume ratios of a measurement sample (1), a measurement sample (2), and a measurement sample (3) were adjusted to S₀:S₁=1:0, S₀:S₁=0:2, and S₀:S₁=1:2, respectively, using the solution (S₀) and the solution (S₁), and a temporal change in fluorescence intensity by ThT staining was measured for each measurement sample. The results are illustrated in FIG. 10. As illustrated in FIG. 10, the fluorescence intensity of the measurement sample (3) (solid line in FIG. 10) was significantly increased as compared with the sum of the measured value of the measurement sample (1) (two-dot chain line in FIG. 10) and the measured value of the measurement sample (2) (long-dashed line in FIG. 10) ((1+2) in FIG. 10 (short-dashed line in FIG. 10)). It was confirmed from this that the protein in the solution (S₀) contributed to the formation of the nanofiber in the presence of the structural protein microbody even though the protein did not form the nanofiber alone.

### Example 2

A structural protein solution was obtained in the same manner as that of Example 1. Next, water was added to the structural protein solution until the concentration of guanidine thiocyanate was 1 M, and then, ethanol was added so that the final concentration was 75 vol % (that is, in order to obtain 4-fold dilution). Therefore, a structural protein microbody was formed in the solution. Centrifugation was performed under conditions of 15,000 g, 10 minutes, and 20° C. using a centrifuge (KUBOTA 3740, manufactured by KUBOTA Manufacturing Corporation) to collect the structural protein microbody as a precipitated fraction. Thereafter, washing with ultrapure water and lyophilization were performed to obtain a structural protein microbody with a yield of 80%.

The obtained structural protein microbody was subjected to the fluorescence intensity measurement by ThT staining, small angle X-ray scattering (SAXS) analysis, and Guinier analysis in the same manner as those of Example 1. As a result, it was confirmed that the structural protein microbody similar to that in Example 1 was obtained.

### Example 3

A powder sample of fibroin having an amino acid sequence set forth in SEQ ID NO: 13 was prepared. To 10 mg of the powder sample, 1 mL of a urea buffer (3 M urea, 10 mM TrisHCl, pH 7.0) was added, and the mixture was shaken (1,800 rpm) for 5 minutes, thereby obtaining a structural protein solution. Next, the structural protein solution was dispensed into a 1.5 mL tube. Thereafter, the structural protein solution was shaken at 3,400 rpm for 30 minutes using a vortex mixer and then was rotated at a high speed to apply a shear stress to the structural protein solution. Therefore, a structural protein microbody was formed in the solution, thereby obtaining a dispersion in which the structural protein microbody was dispersed.

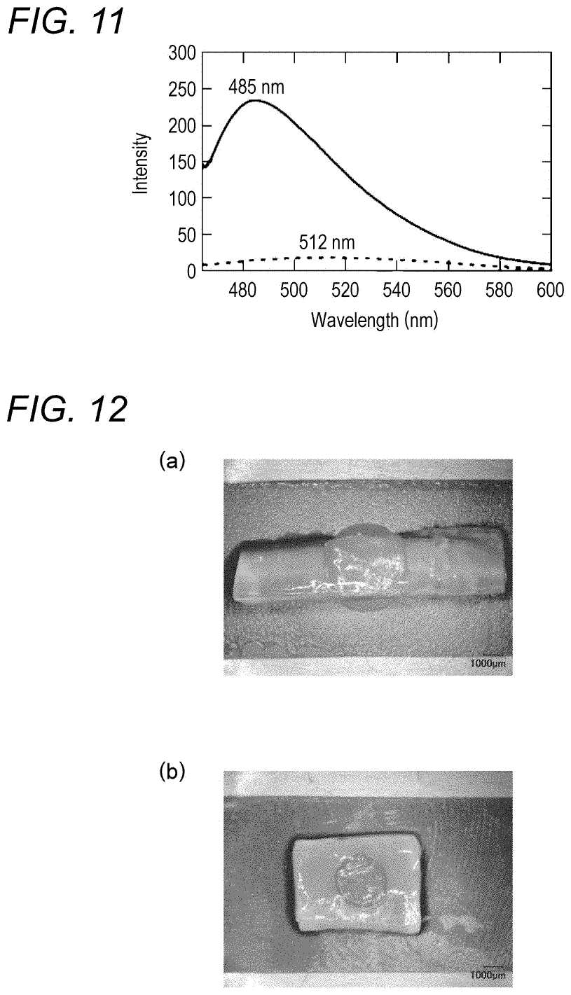

Next, ThT was added to the structural protein microbody dispersion obtained as described above so that a concentration thereof was 4 μM, and a fluorescence intensity measurement was performed under the same conditions as those in Example 1. The result of the fluorescence intensity measurement by ThT staining is indicated by the solid line in FIG. 11. It was confirmed that the structural protein microbody had characteristics because the graph indicated by the solid line in FIG. 11 had a peak within a range of 480 to 500 nm.

### Comparative Example 1

The result of the fluorescence intensity measurement by ThT staining of the protein solution adjusted in the same manner as that of Example 3 except that no shear stress is applied is indicated by the dashed line in FIG. 11. The maximum fluorescence wavelength was 512 nm, and the spectrum showed a broad shape. It was confirmed from the comparison result that the structural protein microbody was obtained by applying a shear stress to a monomer.

### Example 4

A powder sample of fibroin having an amino acid sequence set forth in SEQ ID NO: 13 was prepared. To 5.1 mg of the powder sample of fibroin, 222 μL of a urea buffer (6 M urea, 10 mM trisHCl, 5 mM DTT, pH 7.0) was added, the mixture was shaken (1,800 rpm) for 5 minutes, and then, an ultrasonic treatment (20%, 10 seconds, 4 times, interval of 10 minutes) was performed, thereby completely dissolving the fibroin. Next, the solution in which the fibroin was dissolved was filtered using a filter (Ultrafree-MC-GV, Durapore, PVDF, 0.22 μm) to remove impurities, thereby obtaining a structural protein solution (fibroin solution). Ultraviolet-visible absorption of the obtained structural protein solution was measured by NanoDrop (registered trademark). As a result of the measurement, in an ultraviolet-visible absorption spectrum, an absorption spectrum having a maximum at 280 nm was shown and no remarkable scattering was observed. It was confirmed from the result that the fibroin was not completely dissolved.

Next, the structural protein solution was placed in a dialysis tube (trade name: #D100, manufactured by BioDesign Inc.) formed of a semipermeable membrane, and dialysis was performed using an external solution as a 3 M urea buffer (3 M urea, 10 mM trisHCl, 2.5 mM DTT, pH 7.0) for 24 hours. Thereafter, the external solution was replaced with miliQ (manufactured by Merck Millipore), and dialysis was further performed to obtain a structural protein gel (hydrogel). Here, by such a dialysis operation, a structural protein microbody is formed in the obtained structural protein gel, and the formed structural protein microbody grows into a nanofiber. That is, the structural protein microbody or the nanofiber is contained in the obtained structural protein gel as a fibrous substance.

As illustrated in FIG. 12(a), both ends of the obtained protein gel were fixed in one direction and the protein gel was dried in a form in which an anisotropic stress was applied, thereby obtaining a protein structure.

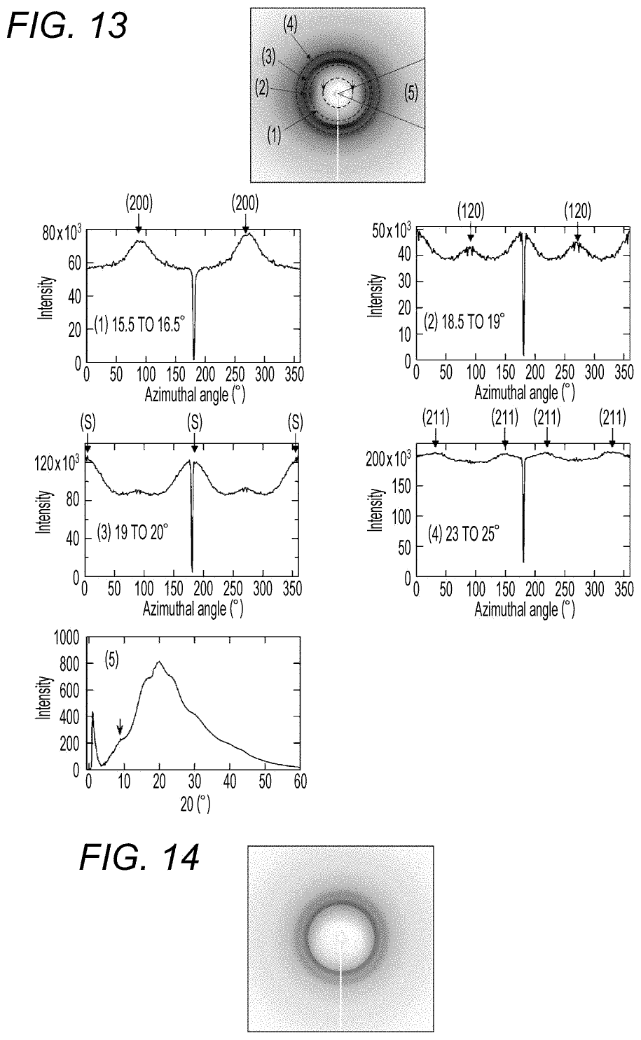

The orientation of the obtained protein structure was confirmed by X-ray diffraction (XRD). Specifically, an X-ray diffraction pattern was obtained using an X-ray generator MicroMAX007 (manufactured by Rigaku Corporation) and a detector R-AXIS-IV (manufactured by Rigaku Corporation) under conditions of an X-ray wavelength of 1.5418 Å (CuKα), room temperature (around 20° C.), a camera length of 80 mm, and an exposure time of 15 minutes. The obtained two-dimensional X-ray diffraction profile is illustrated in FIG. 13. In FIG. 13, each of (1) to (4) illustrates an azimuthal angle distribution of the intensity obtained by circularly multiplying the specific diffraction angle, and (5) illustrates a diffraction intensity profile in a meridian direction. As illustrated in FIG. 13, it was confirmed that the protein nanofiber was highly oriented in the protein structure because the diffraction pattern was arc-shaped and there was a portion observed in a spot shape.

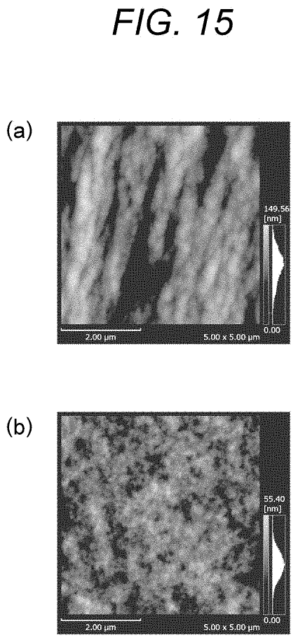

In addition, the orientation of the protein structure was confirmed by an atomic force microscope (AFM). Specifically, an AFM image was obtained by performing a measurement in a dynamic mode using SPM-9700 (manufactured by Shimadzu Corporation) and a cantilever (OMCL-AC 240 TS-R3, manufactured by Olympus Corporation). The obtained AFM image is illustrated in FIG. 15(a). As illustrated in FIG. 15(a), in the AFM image, the fibrous substance oriented in one direction was observed and the high orientation was observed in the protein structure.

### Comparative Example 2

A protein gel was prepared in the same manner as that of Example 4. Then, as illustrated in FIG. 12(b), the protein gel was fixed without regularity and dried to obtain a protein structure.

The obtained protein structure was analyzed by X-ray diffraction (XRD) and was observed with an atomic force microscope (AFM) in the same manner as that of Example 1. The obtained two-dimensional X-ray diffraction profile is illustrated in FIG. 14, and the obtained AFM image is illustrated in FIG. 15(b). As illustrated in FIG. 14, it was confirmed that the diffraction pattern was a vague halo and the orientation was absent in the protein structure. In addition, as illustrated in FIG. 15(b), the orientation of the fibrous substance was not observed in the AFM image.

(Reference Test)

The protein microbody formed in the process of producing a protein gel was subjected to a fluorescence intensity measurement by ThT staining, small angle X-ray scattering (SAXS) analysis, Guinier analysis, and a measurement of an average particle size by a dynamic light scattering method under the following methods.

<Fluorescence Intensity Measurement by ThT Staining>

A measurement sample obtained by dispersing the protein microbody in a dispersion (aqueous solution of 6 M urea, 10 mM TrisHCl, and 5 mM DTT, pH 7.0) at a concentration of 5 mg/mL and further adding 4 μM of ThT was used. The measurement conditions were as follows.

Measuring instrument: JASCO FP-8200 (manufactured by JASCO Corporation), measurement range: 440 to 600 nm, excitation wavelength: 450 nm, scan speed: medium, number of times of measurement: three times

The result of the fluorescence intensity measurement by ThT staining is indicated by A1 in FIG. 3. A1 in FIG. 3 has a peak within a range of 480 to 500 nm, and it was confirmed from this that the protein microbody had a β-sheet structure.

<SAXS Measurement>

A measurement sample obtained by dispersing the protein microbody in a dispersion (aqueous solution of 6 M urea, 10 mM TrisHCl, and 5 mM DTT, pH 7.0) at a concentration of 5 mg/mL and further adding 4 μM of ThT was used.

The measurement conditions were as follows. Measuring apparatus: X-ray small angle scattering measuring apparatus NANO-Viewer (manufactured by Rigaku Corporation), X-ray generator MicroMAX007 (manufactured by Rigaku Corporation), detector PILATUS 200K (manufactured by DECTRIS Ltd.), measurement conditions: X-ray wavelength of 1.5418 Å (CuKα), room temperature (20° C.), exposure time of 30 minutes

After the measurement was performed under the above conditions, circumferential averaging was performed to obtain a one-dimensional profile. A modified Kratky plot was obtained by analyzing the one-dimensional profile using IgorPro software (manufactured by WaveMetrics Inc.). The obtained modified Kratky plot is indicated by A2 (solid line) in FIG. 4. A2 of FIG. 4 has a peak in a region where Q is 0.15 or less. In addition, a change width in a region where Q is 0.15 or more and 0.3 or less is ±10% or less. It was confirmed from this result that the protein microbody had a core portion having a high electron density and a random coil disposed to surround the core portion.

<Guinier Analysis>

Guinier analysis was performed as described in (iii) Aggregate of protein molecules. As a result, it was confirmed that the origin scattering intensity obtained from the first measurement sample group was 20.617, the origin scattering intensity obtained from the second measurement sample group was 7.38, and the protein microbody was an aggregate of three protein molecules.

<Measurement of Average Particle Size by Dynamic Light Scattering Method>

As a measurement sample group, measurement samples were prepared by dispersing protein microbodies in first dispersions (aqueous solution of 6 M urea, 10 mM TrisHCl, and 5 mM DTT, pH 7.0) at concentrations of 2 mg/mL, 4 mg/mL, 6 mg/mL, 8 mg/mL, and 10 mg/mL, respectively. Next, a particle size distribution of each of the measurement samples was measured by a dynamic light scattering method under the following conditions to determine a volume average size. Measuring apparatus: ZETASIZER nano-ZS (manufactured by Malvern Panalytical), measurement temperature: 20° C.

The measurement was performed 5 times for each measurement sample to determine an average value of the obtained measured values. From the concentration and the measured value (average value) of each of the measurement samples, a plot of the average particle size against the concentration was obtained, and 0 concentration extrapolation excluding an intermolecular interaction was performed. The value obtained by the 0 concentration extrapolation was defined as an average particle size of the protein microbodies.

As a result of the measurement, the average particle size of the protein microbodies was 13.225 nm.

### INDUSTRIAL APPLICABILITY

Natural cotton, silk, wool, or the like is an aggregate of nanostructures controlled with high accuracy. On the other hand, according to the present invention, it can be expected to artificially produce a protein nanofiber having a highly controlled structure on an industrial scale. In addition, the protein nanofiber produced by the present invention is also expected to be applied to a cell sheet, a biomolecular device, a filter, spinning, a cosmetic, or the like.

1. A structural protein microbody comprising a structural protein,
wherein the structural protein microbody satisfies at least two of the following (i) to (iii):
(i) a peak is present within a range of 480 to 500 nm in a fluorescence intensity measurement by thioflavin T staining;
(ii) a peak is present in a region where Q is 0.15 or less in a modified Kratky plot of small angle X-ray scattering (SAXS); and
(iii) the structural protein microbody is an aggregate of two or more structural protein molecules.

2. The structural protein microbody according to claim 1, wherein the structural protein microbody satisfies all of (i) to (iii).

3. The structural protein microbody according to claim 1, wherein an average particle size measured by a dynamic light scattering method is 1 to 50 nm.

4. The structural protein microbody according to claim 1, wherein the structural protein microbody satisfies (ii), and a magnitude of the peak is 1.1 times or more greater than an average value in a region where Q is 0.15 or more and 0.3 or less in the modified Kratky plot of small angle X-ray scattering (SAXS).

5. The structural protein microbody according to claim 1, wherein the structural protein microbody satisfies (iii), and an origin scattering intensity normalized by a weight concentration obtained by Guinier analysis is 1.5 times or more greater than an origin scattering intensity of non-aggregated structural protein molecules.

6. The structural protein microbody according to claim 1, wherein the structural protein contains modified fibroin.

7. The structural protein microbody according to claim 6, wherein the structural protein contains modified spider silk fibroin.

8. A method for producing a structural protein microbody, the method comprising:
a first step of obtaining a structural protein solution containing a structural protein and a solubilizing agent; and
a second step of reducing solubility of the structural protein in the structural protein solution to form the structural protein microbody according to claim 1.

9. The method for producing a structural protein microbody according to claim 8, wherein the second step is a step of reducing the solubility by at least one method selected from the group consisting of temperature adjustment, addition of water, addition of a surfactant, addition of an organic solvent, and addition of an inorganic salt.

10. The method for producing a structural protein microbody according to claim 9, wherein the second step is a step of reducing the solubility by two or more methods selected from the group consisting of temperature adjustment, addition of water, addition of a surfactant, addition of an organic solvent, and addition of an inorganic salt.

11. The method for producing a structural protein microbody according to claim 8, wherein the second step is a step of reducing the solubility by applying a shear stress to the structural protein solution.

12. The method for producing a structural protein microbody according to claim 8, wherein the solubilizing agent contains at least one selected from the group consisting of dimethyl sulfoxide, 1,1,1,3,3,3-hexafluoro-2-propanol, guanidine hydrochloride (GuHCl), guanidine thiocyanate, sodium iodide, and perchlorate.

13-15. (canceled)

16. A method for producing a nanofiber, the method comprising:
step A of preparing a protein solution in which a protein is dissolved; and
step B of mixing the protein solution with the structural protein microbody according to claim 1 to obtain a protein nanofiber.

17. The method for producing a nanofiber according to claim 16, wherein the protein solution contains a first solvent, and the first solvent is one selected from the group consisting of an organic solvent, a salt solution, an acidic solution, a basic solution, and a chaotropic solution.

18. The method for producing a nanofiber according to claim 17, wherein the first solvent is one selected from the group consisting of an organic solvent, a salt solution, an acidic solution, and a basic solution.

19. The method for producing a nanofiber according to claim 18, wherein the first solvent is one selected from the group consisting of 1,1,1,3,3,3-hexafluoro-2-propanol and dimethyl sulfoxide.

20. The method for producing a nanofiber according to claim 16, wherein the protein includes a structural protein.

21. The method for producing a nanofiber according to claim 20, wherein the structural protein contains modified fibroin.

22. The method for producing a nanofiber according to claim 21, wherein the structural protein contains modified spider silk fibroin.

23. A method for producing a protein structure, the method comprising:
step (a) of preparing a structural precursor containing a fibrous substance containing a protein; and
step (b) of orienting the fibrous substance in one direction by applying an anisotropic stress to the structural precursor to obtain the protein structure,
wherein the fibrous substance contains at least one of the structural protein microbody according to claim 1 and a protein nanofiber.

24. The method for producing a protein structure according to claim 23, wherein the protein nanofiber is formed by self-organizing the protein using the structural protein microbody as a core.

25. The method for producing a protein structure according to claim 23, wherein the protein nanofiber has an amyloid-like crystal.

26. The method for producing a protein structure according to claim 25, wherein the amyloid-like crystal is oriented perpendicular to an orientation direction of the fibrous substance.

27. (canceled)

28. The method for producing a protein structure according to claim 23, wherein in step (b), the anisotropic stress is applied by fixing both ends of the structural precursor in one direction and drying and shrinking the structural precursor.

29. (canceled)

30. The method for producing a protein structure according to claim 23, wherein the protein contains modified fibroin.

31. (canceled)
